(19) United States
(12) Patent Application Publication
(10) Pub. No.: US 2009/0181041 A1
(43) Pub. Date: Jul. 16, 2009

(54) Production of proteins carrying oligomannose or human-like glycans in yeast and methods of use thereof

(76) Inventors: Jan Holgersson, Huddinge (SE); Anki Gustafsson, Johanneshov (SE); Magnus Sjoholm, (US); Ulrika Rova, (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/284,029

(22) Filed: Sep. 18, 2008

Related U.S. Application Data
(63) Continuation-in-part of application No. 11/626,156, filed on Jan. 23, 2007.
(60) Provisional application No. 60/761,632, filed on Jan. 23, 2006.

(51) Int. Cl. A61K 39/00; C07K 19/00; C12N 1/19; C12P 21/02; C07K 14/47

(52) U.S. Cl. 424/184.1; 530/391.7; 435/254.2; 435/254.23; 435/254.22; 435/254.21; 435/69.1; 530/395

(57) ABSTRACT

Cell lines having genetically modified glycosylation pathways that allow them to carry out a sequence of enzymatic reactions, which mimic the processing of glycoproteins in humans, have been developed. Recombinant proteins expressed in these engineered hosts yield glycoproteins more similar, if not substantially identical, to their human counterparts. The lower eukaryotes, which ordinarily produce high-mannose containing N-glycans, including unicellular and multicellular fungi are modified to produce O-glycans or other structures along human glycosylation pathways. This is achieved using a combination of engineering and/or selection of strains which: do not express certain enzymes which create the undesirable complex structures characteristic of the fungal glycoproteins, which express exogenous enzymes selected either to have optimal activity under the conditions present in the fungi where activity is desired, or which are targeted to an organelle where optimal activity is achieved, and combinations thereof wherein the genetically engineered eukaryote expresses multiple exogenous enzymes required to produce “human-like” glycoproteins.

### RELATED APPLICATIONS

This application is a continuation-in-part of U.S. Ser. No. 11/626,156, filed Jan. 23, 2007, which claims the benefit of U.S. Ser. No. 60/761,632 filed Jan. 23, 2006, the contents of which are each incorporated herein by reference in their entireties.

### FIELD OF THE INVENTION

The present invention relates to the field of glycoprotein production and protein glycosylation engineering in lower eukaryotes, specifically the production of glycoproteins in yeast having oligomannose or humanized O-glycans expressed. The present invention further relates to novel host cells comprising genes encoding enzymes involved in N-acetylgalactosamine transfer to serine or threonine in the peptide chain and production of glycoproteins that are particularly useful as therapeutic agents.

### BACKGROUND OF THE INVENTION

The possibility of producing human recombinant proteins for therapy has revolutionized the treatment of patients with a variety of different diseases. Some proteins, for example insulin that is not glycosylated, can be produced in prokaryotic hosts such as E. coli. Most therapeutic proteins need to be modified by the addition of sugar residues to specific amino acids in the peptide sequence. This glycosylation may be necessary for correct folding of the protein, for long circulation half-times and, in many cases, for optimal activity of the protein. At present, glycosylated proteins are responsible for more than 60% of the annual turnover worldwide for therapeutic proteins. Mammalian cells can produce proteins with a human-like glycosylation, but have other disadvantages like low productivity, with regard to glycosylation heterogenous product formation, and the risk of virus contamination. Yeast cells are robust organisms for industrial fermentation and can be cultivated to high densities in well-defined media.

The glycosylation phenotype of glycoproteins produced in yeast is characterized by oligosaccharides with a high number of mannose residues. N-linked glycans of Pichia are mostly (˜85%) of the high mannose type containing between 8 and 14 mannose residues (Man₈₋₁₄GlcNAcGlcNAc), whereas the rest can be much bigger and contain >30 mannose residues (Man_{>30}GlcNAcGlcNAc). However, even the latter type is much smaller than the N-glycans found on proteins produced in S. cerevisiae (Man_{>50}GlcNAcGlcNAc). O-linked glycans on proteins produced in Pichia are much less well-studied. O-linked glycans with up to five mannose residues in the sugar chain have been described. All of these have been α1,2-linked and they may be phosphorylated.

Recently, a U.S.-based company named GlycoFi was formed in order to commercialize a number of Pichia pastoris strains that had been genetically modified to produce only one well-defined human form of N-linked glycans on proteins expressed in the specific strain. N-linked glycans are important for the parameters mentioned above. However, there have been no attempts in terms of trying to humanize O-glycans on proteins expressed in yeast. A number of biological functions, for example the adhesion of white blood cells to the vascular endothelium during inflammation, are mediated by O-glycans. Recombinant proteins with a defined, human-like O-glycan phenotype can therefore be expected to have a therapeutic value—a value that is mostly confined to the sugar chains themselves. Thus a need exists for a eukaryotic cell that can produce humanized O-linked glycans.

### SUMMARY OF THE INVENTION

The presence of N- and O-linked mannose on yeast produced glycoproteins can, if conjugated to a vaccine antigen, be utilized for specific targeting of the immune system with the aim of creating an enhanced immune response to antigens present on e.g. viruses, bacteria and cancer cells. This can be achieved due to the presence of mannose-binding receptors on certain cells of the human immune system. The mannose-binding receptors include the macrophage mannose receptor (MMR; CD206), which was the first discovered of a family of four mammalian endocytic receptors comprised of an extracellular region containing a cystein-rich (CR) domain, a domain containing fibronectin type two repeats (FNII) and multiple C-type lectin-like carbohydrate recognition domains (CTLD), a transmembrane domain and a short cytoplasmic tail. The family also includes the phospholipase A2 receptor, Endo180 and DEC205 (CD205), but only the MMR and Endo180 have the capacity to bind carbohydrates in a Ca²⁺-dependent manner. They are all type I proteins and contain multiple CTLDs. Another receptor binding high mannose structures is a type II protein on dendritic cells that was first described as a receptor interacting with intercellular adhesion molecule (ICAM)-3 and was therefore named dendritic cell-specific ICAM-3-grabbing nonintegrin (DC-SIGN; CD209).

Both the MMR and DC-SIGN have the capacity to direct internalized antigens into endocytic pathways that result in MHC presentation and subsequent T cell activation. Antibodies specific for MMR or DC-SIGN have, upon coupling to tumor-associated antigens, been shown to stimulate both MHC class I and II-restricted T cell responses. Further, it was recently shown that ovalbumin (OVA) containing either O- or N-glycans, or both, when expressed in the yeast, Pichia pastoris, were more potent than the unmannosylated OVA at inducing OVA-specific CD4⁺ T cell proliferation.

The mannose binding lectin (MBL) is another C-type lectin which selectively binds mannose containing oligosaccharides. The structural arrangement of the CRDs of MBL makes it particularly suitable to bind microbial surfaces with multiple oligosaccharides [17]. MBL is a serum lectin and has functions in activating the complement, in opsonophagocytic processes, modulation of inflammation and promotion of apoptosis [18-20]. The various immunological functions of MMR, DC-SIGN and MBL make them highly interesting targets for recombinant vaccines. Understanding of how the carbohydrate ligand affects internalization and the immunoregulatory properties of MMR, DC-SIGN and MBL is crucial for development of efficient vaccines based on targeting these receptors.

O-linked oligosaccharides of glycoproteins expressed in P. pastoris have shown to be mainly straight Man₂₋₆ polymers [2-23] whereas N-linked glycans typically are of the Man₈₋₁₄GlcNAc₂ type with tri-antennary branched structure [5, 21, 24]. N- and O-linked glycans formed by P. pastoris should therefore be suitable ligands for MMR, DC-SIGN and MBL.

However, for glycoproteins destined for other therapeutic uses than to enhance the immune response towards a specific antigen the nonhuman glycosylation phenotype characterized by oligosaccharides with a high number of mannose residues will trigger an unwanted immune response in humans, leading to a low therapeutic value.

Accordingly, the invention provides fusion proteins containing mannose residues that can be used as aduvants or vaccines. In addition, the invention also provides genetically engineered cells that express humanized glycoproteins.

In one aspect the invention provides a fusion polypeptide containing first polypeptide linked to a second polypeptide. The first polypeptide is mannosylated. By mannosylated is meant that the first polypeptide contains one or more mannose residues. For example, the two, three, four, five, six, seven, eight, nine, ten, fifteen, twenty or more mannose residues per glycan. Optionally, the first polypeptide is hypermannosylated. The mannose residues are N-linked or O-linked

The first polypeptide is a mucin polypeptide. Mucins include for example PSGL-1, MUC1, MUC2, MUC3a, MUC3b, MUC4, MUC5a, MUC5b, MUC5c, MUC6, MUC10, MUC11, MUC12, MUC13, MUC15, MUC16, MUC17, CD34, CD43, CD45, CD96, GlyCAM-1, MAdCAM, or a fragment thereof. The polypeptide is a monomer. Alternatively, the polypeptide is a dimer. Preferably, the polypeptide is for example a P-selectin glycoprotein ligand-1 polypeptide. The polypeptide includes at least a region of a P-selectin glycoprotein ligand-1, such as the extracellular portion of a P-selectin glycoprotein ligand-1. Alternatively, the first polypeptide is an alpha glycoprotein such as an alpha 1-acid glycoprotein (i.e., orosomuciod or AGP) or portion thereof.

The second polypeptide comprises at least a region of an immunoglobulin polypeptide. For example, the second polypeptide includes a region of a heavy chain immunoglobulin polypeptide, such as an F_{c} region or an F_{ab} region.

The Man fusion proteins of the invention bind to mannose-binding receptors with higher affinity than the corresponding wild type protein. For example, the Man fusion protein of the invention is PSGL-1/mIgG_{2b} which binds with higher affinity to a mannose binding receptor than wild type mucin. Alternatively, the Man fusion protein of the invention is AGP-1/mIgG_{2b} which binds with higher affinity to a mannose-binding receptor than wild type alpha glycoprotein.

For example, the Man fusion proteins of the invention bind to a mannose-binding receptor with an affinity ranging from 1 pM to 100 nM, 1 pM to 50 nM, 1 pM to 25 nM, 1 pM to 10 nM, 1 pM to 1 nM, or better. For example, the Man fusion protein of the invention binds to MMR with an affinity of approximately 1 nM to 100 nM, to DC-SIGN with an affinity of approximately 1 nM to 25 nM, and to MBL with an affinity of approximately 1 nM to 50 nM. Mannose-binding receptors include but are not limited to MMR, DC-SIGN and MBL. In a particular embodiment, the Man fusion protein is PSGL-1/mIgG_{2b} and binds to MMR with an affinity of approximately 75 nM, to DC-SIGN with an affinity of approximately 10 nM, and to MBL with an affinity of approximately 5 nM. In another particular embodiment, the Man fusion protein is AGP-1/mIgG_{2b} and binds to MMR with an affinity of approximately 85 nM, to DC-SIGN with an affinity of approximately 20 nM, and to MBL with an affinity of approximately 40 nM.

The mannosylated fusion polypeptides of the invention can be formulated into adjuvant composition. The adjuvant composition can additionally contain a polypeptide carrying Galα1,3Gal epitopes.

Optionally, the mannosylated fusion polypeptide further contains an antigen. The antigen is a for a example a virus, a bacteria or a fungus. For example, the antigen is Hepatitis C, HIV, Hepatitis B, Papilloma virus, Malaria, Tuberculosis, Herpes Simplex Virus, Chlamydia, or Influenza, or, a biological component thereof such as a peptide, protein, lipid carbohydrate, hormone or combination thereof. Alternatively, the antigen is a tumor associated antigen such as a breast, lung, colon, prostate, pancreatic, cervical or melanoma tumor-associated antigen. Optionally, the antigen is operably linked to the mannosylated fusion polypeptide. For example the antigen is covalently linked to the mannosylated fusion polypeptide. Alternatively, the antigen is associated with the mannosylated fusion polypeptide polypeptide non-covalently.

The present invention further relates to an isolated nucleic acid encoding the fusion polypeptide, a vector including this isolated nucleic acid, and a cell comprising this vector. The vector further contains a nucleic acid encoding the antigen polypeptide. Preferably, the nucleic acid encoding the fusion polypeptide is expressed in a yeast cell. For example, the cell is Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pyperi, Pichia stiptis, Pichia methanolica, Pichia sp., Saccharomyces cerevisiae, Saccharomyces sp., Hansenulapolymorpha, Kluyveromyces sp., Candida albicans, Aspergillus nidulans, or Trichoderma reesei. In one embodiment, the invention provides a yeast cell comprising a nucleic acid construct encoding a P-selectin glycoprotein ligand-1 polypeptide or an alpha 1-acid glycoprotein of portion thereof operably linked to at least a region of an immunoglobulin polypeptide, e.g. a heavy chain.

The invention also relates to a method for producing a mannosylated fusion polypeptide having increased glycosylation as compared to a wild type polypeptide. The Man fusion polypeptides may be produced using a prokaryotic or eukaryotic host cell. Preferably, the host cell is a lower eukaryotic cell, e.g., a yeast cell. In a particular embodiment, the lower eukaryotic cell is genetically engineered to produce human-like glycoproteins characterized as having O-linked glycans. The Man fusion polypeptides may be produced by the host cell using fed-batch fermentation using conventional shaker flasks and agitation or a bioreactor (fermentor). A nucleic acid encoding the Man fusion polypeptide of the invention is introduced into said host cell. The host cells are cultured under conditions which permit cell growth until a suitable/desired cell density is reached. For example, a suitable/desired cell density may be 10%, 25%, 30%, 35%, 40%, 45%, 50%, 55%, 60%, 65%, 70%, 75%, 80%, 85%, 90%, 95% or 100% confluency. As used herein, confluency refers to the coverage or proliferation that the cells are allowed over or throughout the culture medium. For example, cell culture conditions which permit cell growth may comprise 29° C. to 37° C. and a neutral or near neutral pH (e.g., pH 6.0-7.0). Suitable cell densities include OD₆₀₀ 5-6 or 1 g DCW/I to approximately 500 g DCWI, (e.g., 10 g DCW/I to 350 g DCW/I, 25 g DCW/I to 250 g DCW/I, or 50 g DCW/I to 150 g DCW/I). Once peak cell density is reached, the cells are induced (i.e., induction phase) to produce the Man fusion polypeptide of the invention. Suitable inducers will depend on the type of fed-batch fermentation technique used (e.g., methanol, glucose, lactose, or IPTG), and are known by the skilled artisan. In a particular embodiment, the host cells are induced under cell culture conditions which permit decreased fragmentation and increased glycosylation of the Man fusion polypeptide as compared to a corresponding wild type polypeptide. Such cell culture conditions may comprise lowering the pH from the neutral/near neutral pH of the culture conditions during the growth phase to create a more acidic environment during the induction phase (e.g., pH<5). In a particular embodiment, lowering the pH from 6.0 during the growth phase to pH 3.5 during the induction phase yields Man fusion polypeptides having decreased fragmentation and increased glycosylation as compared to a corresponding wild type protein. The secreted recombinant proteins are then isolated from the cell culture medium or host cell using methods known to one of ordinary skill in the art.

The invention also features methods of immunization. A subject is immunized by administering to subject in need thereof a mannosylated fusion polypeptide according to the invention and an antigen. The mannosylated fusion polypeptide according to the invention is covalently linked to the antigen. Alternatively, the antigen is associated with the adjuvant polypeptide non-covalently. In a further aspect, the present invention includes a method of preventing or alleviating a symptom of cancer in a subject by identifying a subject in need suffering from or at risk of developing cancer and administering to the subject a mannosylated fusion polypeptide according to the invention and a tumor associated antigen. For example the subject is suffering from or at risk of developing melanoma, breast, lung, colon, prostate, pancreatic, cervical cancer. A subject suffering from or at risk of developing cancer is identified by methods know in the art for the particular disorder.

In a further aspect, the invention provides cell lines having genetically modified glycosylation pathways that allow them to carry out a sequence of enzymatic reactions, which mimic the processing of O-linked glycoproteins in humans. Recombinant proteins expressed in these engineered hosts yield glycoproteins more similar, if not substantially identical, to their human counterparts. The lower eukaryotes, ordinarily produce O-glycans having at least five mannose residue. The cell is unicellular and multicellular fungi such as Pichia pastoris, Hansenulapolymorpha, Pichia stiptis, Pichia methanolica, Pichia sp., Kluyveromyces sp., Candida albicans, Aspergillus nidulans, and Trichoderma reseei, are modified to produce O-glycans or other structures along human glycosylation pathways. This is achieved using a combination of engineering and/or selection of strains which: do not express certain enzymes which create the undesirable complex structures characteristic of the fungal glycoproteins, which express exogenous enzymes selected either to have optimal activity under the conditions present in the fungi where activity is desired, or which are targeted to an organelle where optimal activity is achieved, and combinations thereof wherein the genetically engineered eukaryote expresses multiple exogenous enzymes required to produce “human-like”glycoproteins. Undesirable complex structures include high mannose structure. By high mannose structure is meant eight or more mannose residues per oligosaccharide chain.

The cell is engineered to express one or more exogenous N-acetylgalactosaminyltransferase. Optionally, exogenous enzyme is targeted to the endoplasmic reticulum or Golgi apparatus of the cell.

Optionally, the glycosylation pathway of an eukaryotic microorganism is modified by (a) constructing a DNA library including at least two genes encoding exogenous glycosylation enzymes; (b) transforming the microorganism with the library to produce a genetically mixed population expressing at least two distinct exogenous glycosylation enzymes; (c) selecting from the population a microorganism having the desired glycosylation phenotype. In a preferred embodiment, the DNA library includes chimeric genes each encoding a protein localization sequence and a catalytic activity related to glycosylation. Organisms modified using the method are useful for producing glycoproteins having a glycosylation pattern similar or identical to mammals, especially humans.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a photograph of Western blot analysis of PSGL-1/mIgG_{2b} fusion proteins produced in different clones of Pichia pastoris at 0, 24, 48 and 72 h of induction. The fusion proteins were analysed under non-reducing conditions on 4-12% bis-tris gels, electroblotted onto nitrocellulose membranes and stained with an HRP-conjugated goat anti-mIgG(Fc) antibody.

FIG. 2 is a photograph of Western blot analysis of PSGL-1/mIgG_{2b} fusion proteins produced in different clones (1-5) of Pichia pastoris. The fusion proteins were analysed under non-reducing conditions on 4-12% bis-tris gels, electroblotted onto nitrocellulose membranes and stained with A) an HRP-conjugated goat anti-mIgG (F_{c}) antibody, and B) the lectin Concanavalin A which recognizes mannosylated glycan structures.

FIG. 3 is a photograph of Western blot analysis of AGP-1/mIgG_{2b} fusion proteins (a, lysed cells; b, cell supernatant) produced in different clones (1-4) of Pichia pastoris. The fusion proteins were analysed under non-reducing conditions on 4-12% bis-tris gels, electroblotted onto nitrocellulose membranes and stained with A) an HRP-conjugated goat anti-mIgG (F_{c}) antibody, and B) an anti-AGP-1 antibody. C corresponds to PSGL-1/mIgG_{2b} produced in CHO cells.

FIG. 4 is a graph showing biomass generation for PSGL-1/mIgG_{2b} during the induction phase for bioreactor cultivation (filled squares) and shake flask cultivation (filled diamonds), performed at pH 6.0 and 29° C. for both processes.

FIG. 5 is a graph showing total PSGL-1/mIgG_{2b} protein production for bioreactor cultivation (filled triangles) and shake flask cultivation (filled diamonds) performed at pH 6.0 and 29° C. for both processes.

FIG. 6 is a graph showing AGP-1/mIgG_{2b} specific productivities for bioreactor cultivation (filled triangles) and shake flask cultivation (filled diamonds) performed at pH 6.0 and 29° C.

FIG. 7 depicts an elution profile (UV₂₈₀) of the purification of batch PSGL 7 on a HiTrap MabSelect SuRe column. Fractions A4-A7 were pooled.

FIGS. 8A and 8B are photographs of western blot analysis of PSGL-1/mIgG_{2b} and AGP-mIgG_{2b} produced in Pichia pastoris cells. Membranes were probed with anti-PSGL-1 (8A) or anti-AGP (8B). PSGL-1/mIgG_{2b} purified from batch 7 (lane 1), 8-9, 12 (lane 2), 10-11 (lane 3), AGP-mIgG_{2b} purified from batch 7 (lane 4) and 8 (lane 5), bovine thyroglobulin (lane 6; positive control for Con A), Sialyllactosamine-BSA (lane 7; negative control for Con A), and SLex carrying PSGL-1/mIgG_{2b} purified from glycoengineered CHO cells (lane 8).

FIGS. 8C-8D are photographs of SDS-PAGE gels stained with Pro Q Emerald (8C) followed by Ruby (8D). Supernatant (lanes 1, 3, and 5) and purified PSGL-1/mIgG_{2b} (lanes 2, 4, and 6) from batch 7 (1-2), 8-9,12 (3-4) and 10-11 (5-6). Supernatant (lanes 7 and 9) and purified AGP-mIgG_{2b} (lanes 8 and 10) from batch 7 (7-8) and 8 (9-10).

FIGS. 9A-9C are photographs of western blot analysis of PSGL-1/mIgG_{2b} produced in P. pastoris cells under the following conditions: 9A): Bioreactor cultivation at pH 6.0 and 29° C.; 9B): Shake flask cultivation at pH 6.0 and 29° C.; and 9C) bioreactor cultivation pH 3.5 and 29° C. Membranes were probed with anti-mIgG (F_{c}) antibodies.

FIGS. 10A-10B are photographs of western blot analysis of AGP-1/mIgG_{2b} produced in P. pastoris cells under the following conditions: 9A) Shake flask cultivation at pH 6.0 and 29° C.; and 9B) Bioreactor cultivation (fermentor) at pH 6.0 and 29° C. Membranes were probed with anti-mIgG (F_{c}).

FIG. 11 depicts photographs of western blot analysis of PSGL1-mIgG_{2b} produced in Pichia pastoris (batch 15; pH 3.5). Lane 1: Pp/PSGL1-mIgG_{2b} batch 15, 500 ng (fermentor pH 3.5); Lane 2: Pp/PSGL1-mIgG_{2b} batch 10-11, 500 ng (fermentor, pH 6); Lane 3: Thyroglobulin, bovine, 2 ug; Lane 4: BSA-Lex, 500 ng.

FIG. 12 is a photograph of western blot analysis of PSGL-1/mIgG_{2b} and AGP-mIgG_{2b} produced in Pichia pastoris cells. The membrane was probed with Concanavalin A. The order of the fusion proteins as well as positive and negative controls are the same as in FIGS. 8A and 8B.

FIG. 13 is a photograph of western blot analysis of PSGL-1/mIgG_{2b} produced in Pichia pastoris cells, probed with anti-mIgG antibodies or the mannose-binding lectin Concanavalin A. PSGL-1/mIgG_{2b} fusion proteins were PNGase F-treated to cleave off potential N-glycans (lane 2).

FIG. 14 is an electron spray ionization mass spectrometry (ESI-MS) of O-glycans released from PSGL-1/mIgG_{2b} produced in Pichia pastoris cells.

FIGS. 15A-15F are graphs showing the binding of AGP-1/mIgG_{2b} (a-c) and PSGL-1/mIgG_{2b} (d-f) to MMR (a and d), DC-SIGN (b and e) and MBL (c and f). The concentrations of AGP-1/mIgG_{2b} and PSGL-1/mIgG_{2b} were 0.01 μM, 0.05 μM, 0.1 μM, 0.5 μM, 1 μM and 5 μM.

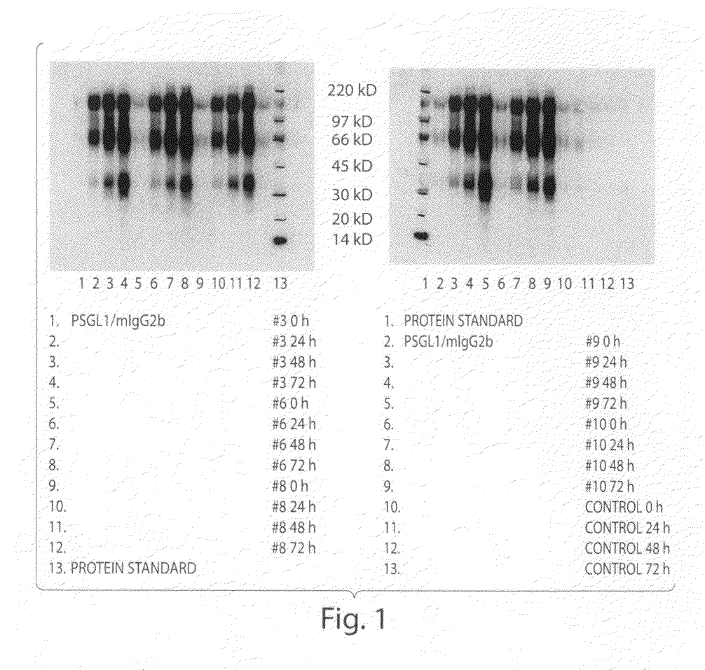

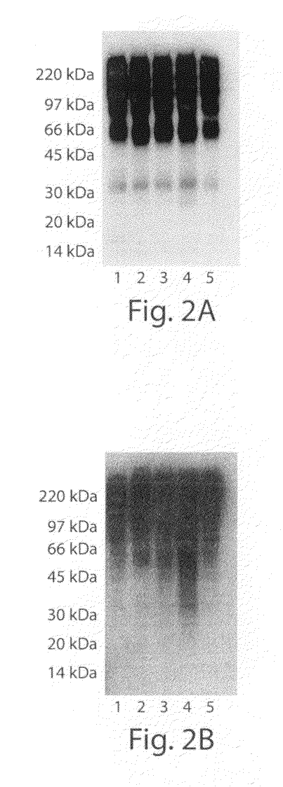

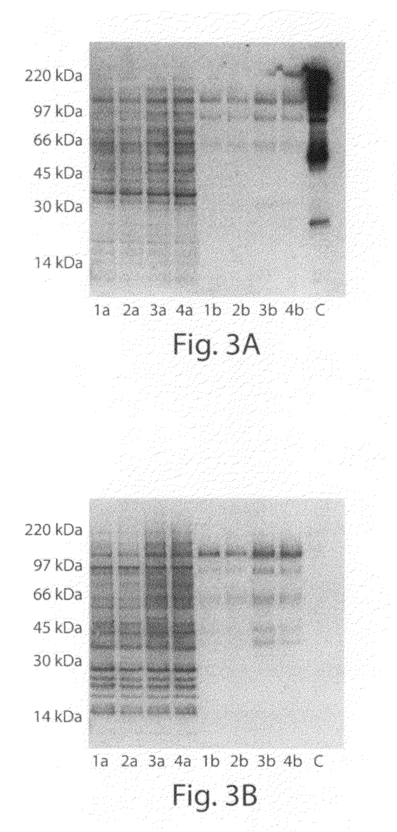

### DETAILED DESCRIPTION

The methods and recombinant lower eukaryotic strains described herein are used to make “humanized glycoproteins”. The recombinant lower eukaryotes are made by engineering lower eukaryotes, which may not express one or more enzymes involved in production of high mannose structures, to express the enzymes required to produce human-like sugars. As used herein, a lower eukaryote is a unicellular or filamentous fungus. As used herein, a “humanized glycoprotein” refers to a protein having attached thereto O-glycans commonly expressed on human mucins and mucin-like proteins (see below), and the synthetic intermediates (which are also useful and can be manipulated further in vitro). This is achieved by cloning in different glycosyltransferases involved in production of O-glycans on human mucins or mucin-like proteins, i.e., enzymes selected to have optimal activity under the conditions present in the organisms at the site where proteins are glycosylated, or by targeting the enzymes to the organelles where activity is desired. In addition, some yeast endogenous mannosyltransferases may be knocked out or knocked down to avoid competition between inserted and endogenous glycosyltransferases. The invention also provides methods in which the high number of mannose residues expressed on glycoproteins produced in yeast are useful in targeting mannose receptors of the human immune system. Thus, in another aspect the invention also provides fusion proteins that are mannosylated, either N- or O-linked, or both.

O-linked glycans are usually attached to the peptide chain through serine or threonine residues. O-linked glycosylation is a true post-translational event and does not require an oligosaccharide precursor for protein transfer. The most common type of O-linked glycans contain an initial GalNAc residue (or Tn epitope), these are commonly referred to as mucin-type glycans. Other O-linked glycans include glucosamine, xylose, galactose, fucose, or mannose as the initial sugar bound to the Ser/Thr residues. O-linked glycoproteins are usually large proteins (>200 kDa) carrying O-glycans that are commonly bianttennary with comparatively less branching than N-glycans. Glycosylation generally occurs in high-density clusters and may contribute as much as 50-80% to the overall mass. O-linked glycans tend to be very heterogeneous, hence they are generally classified by their core structure. Nonelongated O-GlcNAc groups have been recently shown to be related to phosphorylation states and dynamic processing related to cell signaling events in the cell. O-linked glycans are prevalent in most secretory cells and tissues. They are present in high concentrations in the zona pelucida surrounding mammalian eggs and may function as sperm receptors (ZP3 glycoprotein). O-linked glycans are also involved in hematopoiesis, inflammation response mechanisms, and the formation of ABO blood antigens.

Elongation and termination of O-linked glycans is carried out by several glycosyltransferases. One notable core structure is the Galβ(1-3)GalNAc (core 1) sequence that has antigenic properties. Termination of O-linked glycans usually includes Gal, GlcNAc, GalNAc, Fuc, or sialic acid. By far the most common modification of the core Galβ(1-3)GalNAc is mono-, di-, or trisialylation. A less common, but widely distributed O-linked hexasaccharide structure contains β(1-4)-linked Gal and β(1-6)-linked GlcNAc as well as sialic acid.

### Production of Humanized Glycoproteins

Preferably, eukaryotic strains which do not express one or more enzymes involved in the production of N-glycan or O-glycan high mannose structures are used to prevent immunogenic reactions towards possible N- or O-glycans situated on the mucin or mucin-like model fusion protein. These strains can be engineered or be one of the many such mutants already described in yeasts, including a hypermannosylation-minus (OCH1) mutant in Pichia pastoris.

The strains can be engineered one enzyme at a time, or a library of genes encoding potentially useful enzymes can be created, and those strains having enzymes with optimal activities or producing the most “human-like” glycoproteins, selected.

Yeast and filamentous fungi have both been successfully used for the production of recombinant proteins, both intracellular and secreted (Cereghino, J. L. and J. M. Cregg 2000 FEMS Microbiology Reviews 24(1): 45 66; Harkki, A., et al. 1989 Bio-Technology 7(6): 596; Berka, R. M., et al. 1992 Abstr. Papers Amer. Chem. Soc. 203: 121-BIOT; Svetina, M., et al. 2000 J. Biotechnol. 76(2 3): 245 251).

Although glycosylation in yeast and fungi is very different than in humans, some common elements are shared. The first step of N-glycosylation, the transfer of the core oligosaccharide structure to the nascent protein, is highly conserved in all eukaryotes including yeast, fungi, plants and humans. Subsequent processing of the core oligosaccharide, however, differs significantly in yeast and involves the addition of several mannose sugars. This step is catalyzed by mannosyltransferases residing in the Golgi (e.g. OCH1, MNT1, MNN1, etc.), which sequentially add mannose sugars to the core oligosaccharide. The resulting structure is undesirable for the production of humanoid proteins and it is thus desirable to reduce or eliminate mannosyl transferase activity. Mutants of S. cerevisiae, deficient in mannosyl transferase activity (e.g. och1 or mnn9 mutants) have shown to be non-lethal and display a reduced mannose content in the oligosacharide of yeast glycoproteins. Other oligosacharide processing enzymes, such as mannosylphophate transferase may also have to be eliminated depending on the host's particular endogenous glycosylation pattern. After reducing undesired endogenous glycosylation reactions the formation of complex O-glycans is engineered into the host system. This requires the stable expression of several enzymes and sugar-nucleotide transporters. Moreover, one has to locate these enzymes in a fashion such that a sequential processing of the maturing glycosylation structure is ensured.

The methods described herein are useful for producing glycoproteins, especially glycoproteins used therapeutically in humans. Such therapeutic proteins are typically administered by injection, orally, pulmonary, or by other means.

The initial addition of a GalNAc to serine or threonine in the peptide sequence is performed by UDP-GalnAc-polypeptide N-acetylgalactosaminyltransferases (ppGalnAcTs). Fourteen ppGalNAcTs have been identified to date, ten of them in humans. The different ppGalNAcTs seem to be differently expressed in tissues, some overlapping and with a more ubiquitous expression than others. Further, individual ppGalNAcTs seem to have different peptide substrate specificities. ppGalNAcT1 is highly inhibited by neighboring glycosylated residues, while neighboring peptide residues seem to have minor influence on its activity, thus suggesting that ppGalNAcT1 is responsible for the initial glycosylation of peptides. The core 1 structure is generated by a β1,3-galactosyltransferase (C1 β3GalT). To days date, only one gene encoding a C1 β3GalT enzyme has been cloned. The C1 β3GalT is ubiquitously expressed in mammals and has been shown to require a chaperone for its activity. The core 2 structure is produced by the addition of a GlcNAc in a β1,6-linkage to core 1. Three core 2 N-acetylglucosaminyltransferases (C2 GnTs) have been cloned. C2 GnT-I has a widespread occurrence. In particular, it is highly expressed in spleen, which indicates a strong expression in B-cells. C2 GnT-II transcripts are highly expressed in mucin producing organs, such as the colon, small intestine, trachea, and stomach. This enzyme was shown to also have core 4 branching activity, which is not seen for C2 GnT-I. A third C2 GnT (C2 GnT-HD has been cloned that, like C2 GnT-I, have mainly core 2 branching activity. Northern blot analysis revealed the transcript of this enzyme to be highly expressed in thymus, while only low levels could be detected in other organs. Core 3 is synthesized by C3 GnT-VI, which adds a GlcNAc in a β1,3-linkage to the innermost GalNAc. Thus, this enzyme competes with the C1 β3GalT. The core 3 structure can then be elongated into type 4 by the addition of a GlcNAc in a β1,6-linkage to the peptide-linked GalNAc. The different core structures can be produced by expression of the above mentioned enzymes in yeast cells.

O-glycan terminal determinants vary even further on human glycoproteins. The majority of serum and membrane glycoproteins express mono- or disialylated core 1 structures. However, longer O-glycans terminating in e.g. blood group (ABH) and Lewis antigens can be found. Especially, such structures are present on different cells of the hemopioetic lineage, e.g. sialyl Lewis x (SLe^{x}) on P-selectin glycoproteins ligand-1 (PSGL-1) expressed on leukocytes and interacting with P-selectin present on activated endothelial cells. Also, O-glycans may express α1,4-linked GlcNAc, a structure unique for this group of glycans. The terminal determinants are often expressed on lactosamine (LacNAc), or even branched repetitive LacNAc units (i and I antigens). Both branches of the trisaccharide cores (core 2 and 4) may be elongated, but the C6-branch is generally preferred over the C3-branch. The genes of the glycosyltransferases responsible for the production of above mentioned terminal determinants have been cloned and can therefore be inserted into yeast cells in order to promote the production of human-like O-glycans.

The method described herein may be used to engineer the glycosylation pattern of a wide range of lower eukaryotes (e.g. Hansenula polymorpha, Pichia stiptis, Pichia methanolica, Pichia sp, Kluyveromyces sp, Candida albicans, Aspergillus nidulans, Trichoderma reseei etc.). Pichia pastoris is used as an example. Similar to other lower eukaryotes, P. pastoris produces Man₉GlcNAc₂ structures in the ER. Glycoproteins produced in yeast cells modified as described above will express human-like O-glycans. However, the chosen proteins may also contain one or more N-glycosylation sites. In order to avoid the expression of high-mannose N-glycans on the produced glycoproteins it is of importance to eliminate the ability of the fungus to hypermannosylate existing Man₉GlcNAc₂ structures. This can be achieved by either selecting for a fungus that does not hypermannosylate, or by genetically engineering such a fungus.

Genes that are involved in this process have been identified in Pichia pastoris and by creating mutations in these genes one is able to reduce the production of “undesirable” glycoforms. Such genes can be identified by homology to existing mannosyltransferases (e.g. OCH1, MNN4, MNN6, MNN1), found in other lower eukaryotes such as C. albicans, Pichia angusta or S. cerevisiae or by mutagenizing the host strain and selecting for a phenotype with eliminated or reduced mannosylation. Alternatively, one may be able to complement particular phenotypes in related organisms. For example, in order to obtain the gene or genes encoding 1,6-mannosyltransferase activity in P. pastoris, one would carry out the following steps. OCH1 mutants of S. cerevisiae are temperature sensitive and are slow growers at elevated temperatures. One can thus identify functional homologues of OCH1 in P. pastoris by complementing an OCH1 mutant of S. cerevisiae with a P. pastoris DNA or cDNA library. Such mutants of S. cerevisiae may be found e.g., see the Saccharomyces genome link at the Stanford University website and are commercially available. Mutants that display a normal growth phenotype at elevated temperature, after having been transformed with a P. pastoris DNA library, are likely to carry an OCH1 homologue of P. pastoris. Such a library can be created by partially digesting chromosomal DNA of P. pastoris with a suitable restriction enzyme and after inactivating the restriction enzyme ligating the digested DNA into a suitable vector, which has been digested with a compatible restriction enzyme. Suitable vectors are pRS314, a low copy (CEN6/ARS4) plasmid based on pBluescript containing the Trp1 marker (Sikorski, R. S., and Hieter, P., 1989, Genetics 122, pg 19 27) or pFL44S, a high copy (2 .beta.) plasmid based on a modified pUC19 containing the URA3 marker (Bonneaud, N., et al., 1991, Yeast 7, pg. 609 615). Such vectors are commonly used by academic researchers or similar vectors are available from a number of different vendors such as Invitrogen (Carlsbad, Calif.), Pharmacia (Piscataway, N.J.), New England Biolabs (Beverly, Mass.). Examples are pYES/GS, 2.beta origin of replication based yeast expression plasmid from Invitrogen, or Yep24 cloning vehicle from New England Biolabs. After ligation of the chromosomal DNA and the vector one may transform the DNA library into strain of S. cerevisiae with a specific mutation and select for the correction of the corresponding phenotype. After sub-cloning and sequencing the DNA fragment that is able to restore the wild-type phenotype, one may use this fragment to eliminate the activity of the gene product encoded by OCHi in P. pastoris.

Alternatively, if the entire genomic sequence of a particular fungus of interest is known, one may identify such genes simply by searching publicly available DNA databases, which are available from several sources such as NCBI, Swissprot etc. For example by searching a given genomic sequence or data base with a known 1,6 mannosyltransferase gene (OCH1) from S. cerevisiae, one can able to identify genes of high homology in such a genome, which a high degree of certainty encodes a gene that has 1,6 mannosyltransferase activity. Homologues to several known mannosyltransferases from S. cerevisiae in P. pastoris have been identified using either one of these approaches. These genes have similar functions to genes involved in the mannosylation of proteins in S. cerevisiae and thus their deletion may be used to manipulate the glycosylation pattern in P. pastoris or any other fungus with similar glycosylation pathways.

The creation of gene knock-outs, once a given target gene sequence has been determined, is a well-established technique in the yeast and fungal molecular biology community, and can be carried out by anyone of ordinary skill in the art (R. Rothsteins, (1991) Methods in Enzymology, vol. 194, p. 281). In fact, the choice of a host organism may be influenced by the availability of good transformation and gene disruption techniques for such a host. If several mannosyltransferases have to be knocked out, the method developed by Alani and Kleckner allows for the repeated use of the URA3 markers to sequentially eliminate all undesirable endogenous mannosyltransferase activity. This technique has been refined by others but basically involves the use of two repeated DNA sequences, flanking a counter selectable marker. For example: URA3 may be used as a marker to ensure the selection of a transformants that have integrated a construct. By flanking the URA3 marker with direct repeats one may first select for transformants that have integrated the construct and have thus disrupted the target gene. After isolation of the transformants, and their characterization, one may counter select in a second round for those that are resistant to 5′FOA. Colonies that able to survive on plates containing 5′FOA have lost the URA3 marker again through a crossover event involving the repeats mentioned earlier. This approach thus allows for the repeated use of the same marker and facilitates the disruption of multiple genes without requiring additional markers.

Eliminating specific mannosyltransferases, such as 1,6 mannosyltransferase (OCH1), mannosylphosphate transferases (MNN4, MNN6, or genes complementing lbd mutants) in P. pastoris, allows for the creation of engineered strains of this organism which synthesize primarily Man₈GlcNAc₂ and thus can be used to further modify the glycosylation pattern to more closely resemble more complex human glycoform structures. A preferred embodiment of this method utilizes known DNA sequences, encoding known biochemical glycosylation activities to eliminate similar or identical biochemical functions in P. pastoris, such that the glycosylation structure of the resulting genetically altered P. pastoris strain is modified.

Most enzymes that are active in the ER and Golgi apparatus of S. cerevisiae have pH optima that are between 6.5 and 7.5. All previous approaches to reduce mannosylation by the action of recombinant mannosidases have concentrated on enzymes that have a pH optimum around pH 5.0 (Martinet et al., 1998, and Chiba et al., 1998), even though the activity of these enzymes is reduced to less than 10% at pH 7.0 and thus most likely provide insufficient activity at their point of use, the ER and early Golgi of P. pastoris and S. cerevisiae. A preferred process utilizes an α-mannosidase in vivo, where the pH optimum of the mannosidase is within 1.4 pH units of the average pH optimum of other representative marker enzymes localized in the same organelle(s). The pH optimum of the enzyme to be targeted to a specific organelle should be matched with the pH optimum of other enzymes found in the same organelle, such that the maximum activity per unit enzyme is obtained.

When one attempts to trim high mannose structures to yield Man₅GlcNAc₂ in the ER or the Golgi apparatus of S. cerevisiae, one may choose any enzyme or combination of enzymes that (1) has/have a sufficiently close pH optimum (i.e. between pH 5.2 and pH 7.8), and (2) is/are known to generate, alone or in concert, the specific isomeric Man₅GlcNAc₂ structure required to accept subsequent addition of GlcNAc by GnT I. Any enzyme or combination of enzymes that has/have shown to generate a structure that can be converted to Man₅GlcNAc₂ by GnT I in vitro would constitute an appropriate choice. This knowledge may be obtained from the scientific literature or experimentally by determining that a potential mannosidase can convert Man₈GlcNAc₂ to Man₅GlcNAc₂-PA and then testing, if the obtained Man₅GlcNAc₂-PA structure can serve a substrate for GnT I and UDP-GlcNAc to give GlcNAcMan.sub.5GlcNAc.sub.2 in vitro. For example, mannosidase IA from a human or murine source would be an appropriate choice.

Previous approaches to reduce mannosylation by the action of cloned exogenous mannosidases have failed to yield glycoproteins having a sufficient fraction (e.g. >27 mole %) of O-glycans (Martinet et al., 1998, and Chiba et al., 1998). These enzymes should function efficiently in ER or Golgi apparatus to be effective in converting nascent glycoproteins.

A second step of the process involves the sequential addition of sugars to the nascent carbohydrate structure by engineering the expression of glucosyltransferases into the Golgi apparatus. This process first requires the functional expression of GnT I in the early or medial Golgi apparatus as well as ensuring the sufficient supply of UDP-N-acetyl-D-galactosaminide.

Since the ultimate goal of this genetic engineering effort is a robust protein production strain that is able to perform well in an industrial fermentation process, the integration of multiple genes into the fungal chromosome involves careful planing. The engineered strains are transformed with a range of different genes, and these genes will have to be transformed in a stable fashion to ensure that the desired activity is maintained throughout the fermentation process. Any combination of the following enzyme activities will have to be engineered into the fungal protein expression host: sialyltransferases, mannosidases, fucosyltransferases, galactosyltransferases, glucosyltransferases, GlcNAc transferases, ER and Golgi specific transporters (e.g. syn and antiport transporters for UDP-galactose and other precursors), other enzymes involved in the processing of oligosaccharides, and enzymes involved in the synthesis of activated oligosaccharide precursors such as UDP-galactose, CMP-N-acetylneuraminic acid. At the same time a number of genes which encode enzymes known to be characteristic of non-human glycosylation reactions, will have to be deleted.

Glycosyltransferases and mannosidases line the inner (luminal) surface of the ER and Golgi apparatus and thereby provide a “catalytic” surface that allows for the sequential processing of glycoproteins as they proceed through the ER and Golgi network. In fact the multiple compartments of the cis, medial, and trans Golgi and the trans-Golgi Network (TGN), provide the different localities in which the ordered sequence of glycosylation reactions can take place. As a glycoprotein proceeds from synthesis in the ER to full maturation in the late Golgi or TGN, it is sequentially exposed to different glycosidases, mannosidases and glycosyltransferases such that a specific carbohydrate structure may be synthesized. Much work has been dedicated to revealing the exact mechanism by which these enzymes are retained and anchored to their respective organelle. The evolving picture is complex but evidence suggests that stem region, membrane spanning region and cytoplasmic tail individually or in concert direct enzymes to the membrane of individual organelles and thereby localize the associated catalytic domain to that locus.

Targeting sequences are well known and described in the scientific literature and public databases, as discussed in more detail below with respect to libraries for selection of targeting sequences and targeted enzymes.

### Mannosylated Fusion Proteins

Also included in the invention are fusion proteins carrying N- or O-linked, or both, oligomannose structures. The fusion proteins of the invention are useful in enhancing the response towards specific antigens. This can be achieved by conjugation of the mannosylated fusion protein to vaccine antigens.

For safety reasons many vaccines are composed of recombinant antigens of the pathogen instead of live-attenuated or inactivated forms. Such vaccines often suffer from poor immunogenicity and require adjuvants for adequate immune responses [1]. Mannosylation of model antigens has shown to improve antigen presentation and enhance both humoral and cytolytic T-lymphocyte (CTL) responses in mouse models [2-4]. Recombinant glycoproteins expressed in yeast display high mannose content [5] which suggests their use in production of recombinant mannosylated vaccines. In support of this, antigens glycosylated by the methylotrophic yeast Pichia pastoris have been shown to enhance CD4⁺ and CD8⁺ T-cell responses compared to non-glycosylated antigens [6, 7].

The fusion proteins of the invention carrying N- or O-linked, or both, oligomannose structures, will target the vaccine antigen to macrophages and dendritic cells via binding to mannose-binding receptors, thereby increasing the immunogenicity of various vaccine constituents. Accordingly, the mannosylated fusion proteins of the invention are useful as vaccine adjuvants. Such targeting is also useful for various imaging applications.

The mannose-binding receptors include the macrophage mannose receptor (MMR; CD206), which was the first discovered of a family of four mammalian endocytic receptors comprised of an extracellular region containing a cystein-rich (CR) domain, a domain containing fibronectin type two repeats (FNII) and multiple C-type lectin-like carbohydrate recognition domains (CTLD), a transmembrane domain and a short cytoplasmic tail. The family also include the phospholipase A2 receptor, Endo180 and DEC205 (CD205), but only the MMR and Endo180 have the capacity to bind carbohydrates in a Ca²⁺-dependent manner. They are all type I proteins and contain multiple CTLDs. Another receptor binding high mannose structures is a type II protein on dendritic cells that was first described as a receptor interacting with intercellular adhesion molecule (ICAM)-3 and was therefore named dendritic cell-specific ICAM-3-grabbing nonintegrin (DC-SIGN; CD209).

Both the MMR and DC-SIGN have the capacity to direct internalized antigens into endocytic pathways that result in MHC presentation and subsequent T cell activation [4, 8, 10]. However, the binding patterns are different for MMR and DC-SIGN. In MMR, high affinity binding and endocytosis of glycoconjugates are mediated through several carbohydrate recognition domains (CRDs) within a single receptor whereas the carbohydrate binding domain in DC-SIGN resides in a single CRD at its extracellular C-terminal [11, 12]. Whereas MMR preferentially binds terminal mannoses, DC-SIGN has suggested selectivity towards high mannose N-glycans [13, 14]. Besides their antigen internalization capabilities and roles in antigen presentation, MMR and DC-SIGN could mediate several immunoregulatory functions important for DC-migration, macrophage- and DC-expression of pro- or anti-inflammatory mediators and co-stimulatory signals [12, 15, 16]. There are indications that the oligosaccharide part of the glycosylated antigen plays an important role as to the type and strength of the immune response elicited by targeting MMR or DC-SIGN [3, 7]. This suggests that carbohydrates of glycoproteins could be tailored for specific responses. Antibodies specific for MMR or DC-SIGN have upon coupling to tumor-associated antigens been shown to stimulate both MHC class I and II-restricted T cell responses. Further, it was recently shown that ovalbumin (OVA) containing either O- or N-glycans, or both, when expressed in the yeast, Pichia pastoris, were more potent than the unmannosylated OVA at inducing OVA-specific CD4⁺ T cell proliferation.

The mannose binding lectin (MBL) is another C-type lectin which selectively binds mannose containing oligosaccharides. The structural arrangement of the CRDs of MBL makes it particularly suitable to bind microbial surfaces with multiple oligosaccharides [17]. MBL is a serum lectin and has functions in activating the complement, in opsonophagocytic processes, modulation of inflammation and promotion of apoptosis [18-20]. The various immunological functions of MMR, DC-SIGN and MBL make them highly interesting targets for recombinant vaccines. Understanding of how the carbohydrate ligand affects internalization and the immunoregulatory properties of MMR, DC-SIGN and MBL is crucial for development of efficient vaccines based on targeting these receptors.

The invention provides glycoprotein-immunoglobulin fusion proteins (referred to herein as “Man fusion protein or Man fusion peptides”) containing multiple mannose epitopes.

The Man fusion proteins or Man fusion peptides are more efficient on a carbohydrate molar basis in inhibiting mannose receptor-ligand binding as compared to free saccharides. The reason for this is most likely the multivalent presentation of the mannosylated glycans as compared to monovalent free oligosaccharides.

The mannosylated fusion peptide inhibits 2, 4, 10, 20, 50, 80, 100 or more-fold greater number of mannose receptor-ligand binding to an equivalent amount of free saccharides.

In various aspects the invention provides fusion proteins that include a first polypeptide containing at least a portion of a glycoprotein, e.g., a mucin polypeptide or an alpha-globulin polypeptide, operatively linked to a second polypeptide. As used herein, a “fusion protein” or “chimeric protein” includes at least a portion of a glycoprotein polypeptide operatively linked to a non-mucin polypeptide.

A “mucin polypeptide” refers to a polypeptide having a mucin domain. The mucin polypeptide has one, two, three, five, ten, twenty or more mucin domains. The mucin polypeptide is any glycoprotein characterized by repetitive amino acid sequences, called tandem repeats, substituted with O-glycans. For example, a mucin polypeptide has every second or third amino acid being a serine or threonine. The mucin polypeptide is a secreted protein. Alternatively, the mucin polypeptide is a cell surface protein.

Mucin domains are rich in the amino acids threonine, serine and proline, where the oligosaccharides are linked via N-acetylgalactosamine to the hydroxy amino acids (O-glycans). A mucin domain comprises or alternatively consists of an O-linked glycosylation site. A mucin domain has 1, 2, 3, 5, 10, 20, 50, 100 or more O-linked glycosylation sites. A mucin polypeptide has 50%, 60%, 80%, 90%, 95% or 100% of its mass due to the glycan. A mucin polypeptide is any polypeptide encoded for by a MUC gene (i.e., MUC1, MUC2, MUC3a, MUC3b, MUC4, MUC5a, MUC5b, MUC5c, MUC6, MUC10, MUC11, MUC12, MUC13, MUC15, MUC16, and MUC17). Alternatively, a mucin polypeptide is P-selectin glycoprotein ligand 1 (PSGL-1), CD34, CD43, CD45, CD96, GlyCAM-1, MAdCAM, or red blood cell glycophorins. Preferably, the mucin is PSGL-1.

An “alpha-globulin polypeptide” refers to a serum glycoprotein. Alpha-globulins include for example, enzymes produced by the lungs and liver, and haptoglobin, which binds hemoglobin together. An alpha-globulin is an alpha₁ or an alpha₂ globulin. Alpha₁ globulin is predominantly alpha₁antitrypsin, an enzyme produced by the lungs and liver. Alpha₂ globulin, which includes serum haptoglobin, is a protein that binds hemoglobin to prevent its excretion by the kidneys. Other alphaglobulins are produced as a result of inflammation, tissue damage, autoimmune diseases, or certain cancers. Preferably, the alpha-globulin is alpha-1-acid glycoprotein (i.e., orosomucoid).

A “non-mucin polypeptide” refers to a polypeptide of which at least less than 40% of its mass is due to glycans. As used herein, the following definitions are supplied in order to facilitate the understanding of this case. To the extent that the definitions vary from meanings known to those skilled in the art, the definitions below control.

By “biological component” is meant any compound created by or associated with a cell, tissue, bacteria, virus, or other biological entity, including peptides, proteins, lipids, carbohydrates, hormones, or combinations thereof.

By “adjuvant compound” is meant any compound that increases an immunogenic response or the immunogenicity of an antigen or vaccine.

By “antigen” is meant any compound capable of inducing an immunogenic response.

By “immunoglobulin” is meant any polypeptide or protein complex that is secreted by plasma cells and that functions as an antibody in the immune response by binding with a specific antigen. Immunoglobulins as used herein include IgA, IgD, IgE, IgG, and IgM. Regions of immunoglobulins include the F_{c} region and the F_{ab} region, as well as the heavy chain or light chain immunoglobulins.

By “antigen presentation” is meant the expression of an antigen on the surface of a cell in association with one or more major hisocompatability complex class I or class II molecules. Antigen presentation is measured by methods known in the art. For example, antigen presentation is measured using an in vitro cellular assay as described in Gillis, et al., J. Immunol. 120: 2027 1978.

By “immunogenicity” is meant the ability of a substance to stimulate an immune response. Immunogenicity is measured, for example, by determining the presence of antibodies specific for the substance. The presence of antibodies is detected by methods known in the art, for example, an ELISA assay.

By “immune response” or “immunogenic response” is meant a cellular activity induced by an antigen, such as production of antibodies or presentation of antigens or antigen fragments.

By “proteolytic degradation” is meant degradation of the polypeptide by hydrolysis of the peptide bonds. No particular length is implied by the term “peptide.” Proteolytic degradation is measured, for example, using gel electrophoresis.

The “cell” includes any cell capable of antigen presentation. For example, the cell is a somatic cell, a B-cell, a macrophage or a dendritic cell.

Within a Man fusion protein of the invention the mucin polypeptide corresponds to all or a portion of a mucin or mucin-type protein. A Man fusion protein comprises at least a portion of a mucin or mucin-type protein. “At least a portion” is meant that the mucin polypeptide contains at least one mucin domain (e.g., an O-linked glycosylation site). The mucin protein comprises the extracellular portion of the polypeptide. For example, the mucin polypeptide comprises the extracellular portion of PSGL-1.

A Man fusion protein comprises at least a portion of an alpha globulin polypeptide. The alpha globulin polypeptide can corresponds to all or a portion of an alpha globulin polypeptide. “At least a portion” is meant that the alpha globulin polypeptide contains at least one N-linked glycosylation site.

The first polypeptide is glycosylated by one or more glycotransferases. The first polypeptide is glycosylated by 2, 3, 4, 5 or more glycotransferases. Glycosylation is sequential or consecutive. Alternatively glycosylation is concurrent or random. By glycosyltransferases are referred to glycosyltransferases known to be involved in the production of N- or O-linked glycan chains, both mannosylated structures and human-like glycans. The first polypeptide contains greater that 40%, 50%, 60%, 70%, 80%, 90% or 95% of its mass due to carbohydrate

Within the fusion protein, the term “operatively linked” is intended to indicate that the first and second polypeptides are chemically linked (most typically via a covalent bond such as a peptide bond) in a manner that allows for O-linked and/or N-linked glycosylation of the first polypeptide. When used to refer to nucleic acids encoding a fusion polypeptide, the term operatively linked means that a nucleic acid encoding the mucin/mucin-type or alpha globulin polypeptide and the non-mucin polypeptide are fused in-frame to each other. The non-mucin polypeptide can be fused to the N-terminus or C-terminus of the mucin/mucin-type or alpha globulin polypeptide.

The Man fusion proteins of the invention include, are homodimeric chimeric glycoproteins with different potential for N- and O-linked glycosylation. A Man fusion protein of the invention includes, without limitation, PSGL-1/mIgG_{2b}. PSGL-1/mIgG_{2b} is a mucin-like protein with, as a dimer, 106 potential sites for O-linked glycosylation and six potential sites for N-linked glycosylation. The Man fusion protein of the invention includes, without limitation, AGP-1/mIgG_{2b}. AGP-1/mIgG_{2b} has, as a dimer, twelve potential sites for N-linked glycosylation [25, 26]. By displaying multiple ligands in various combinations for both MMR, DC-SIGN and MBL, PSGL-1/mIgG_{2b} and AGP-1/mIgG_{2b} can target these receptors with high affinity by engaging multiple CRDs of MMR and MBL or multiple/oligomerized DC-SIGN. Hence, both recombinant proteins have the potential to target the receptors and promote enhanced immune responses in vivo.

The Man fusion proteins of the invention bind to mannose-binding receptors with higher affinity than the corresponding wild type protein. For example, without limitation, the Man fusion protein of the invention binds to a mannose-binding receptor with higher affinity than a wild type mucin or alpha glycoprotein polypeptide. The Man fusion proteins bind to a mannose-binding receptor with affinity ranging from 1 pM to 100 nM, 1 pM to 50 nM, 1 pM to 25 nM, 1 pM to 10 nM, 1 pM to 1 nM, or better. For example, the Man fusion protein of the invention binds to MMR with an affinity of approximately 1 nM to 100 nM, to DC-SIGN with an affinity of approximately 1 nM to 25 nM, and to MBL with an affinity of approximately 1 nM to 50 nM. Mannose-binding receptors include but are not limited to MMR, DC-SIGN and MBL. In a particular embodiment, the Man fusion protein is PSGL-1/mIgG_{2b} and binds to MMR with an affinity of approximately 75 nM, to DC-SIGN with an affinity of approximately 10 nM, and to MBL with an affinity of approximately 5 nM. In another particular embodiment, the Man fusion protein is AGP-1/mIgG_{2b} and binds to MMR with an affinity of approximately 85 nM, to DC-SIGN with an affinity of approximately 20 nM, and to MBL with an affinity of approximately 40 nM.

The Man fusion protein is linked to one or more additional moieties. For example, the Man fusion protein may additionally be linked to a GST fusion protein in which the Man fusion protein sequences are fused to the C-terminus of the GST (i.e., glutathione S-transferase) sequences. Such fusion proteins can facilitate the purification of the Man fusion protein. Alternatively, the Man fusion protein may additionally be linked to a solid support. Various solid supports are known to those skilled in the art. Such compositions can facilitate removal of anti-blood group antibodies. For example, the Man fusion protein is linked to a particle made of, e.g., metal compounds, silica, latex, polymeric material; a microtiter plate; nitrocellulose, or nylon or a combination thereof. The Man fusion proteins linked to a solid support are used as an absorber to remove microbes, bacterial toxins or other Man-binding proteins from biological sample, such as gastric tissue, blood or plasma.

Optionally, the Man fusion protein is linked to an antigen to form a vaccine. An “antigen” includes any compound to which an immune response is desired. An antigen includes any substance that, when introduced into the body, stimulates an immune response, such as the production of an antibody from a B cell, activation and expansion of T cells, and cytokine expression (e.g., interleukins). By a “B cell” or “B lymphocyte” is meant an immune cell that, when activated, is responsible for the production of antibodies. By a “T cell” or “T lymphocyte” is meant a member of a class of lymphocytes, further defined as cytotoxic T cells and helper T cells. T cells regulate and coordinate the overall immune response, identifying the epitopes that mark the antigens, and attacking and destroying the diseased cells they recognize as foreign. Antigens include for example, toxins, bacteria, foreign blood cells, and the cells of transplanted organs. Preferably, the antigen is Hepatitis C, HIV, Hepatitis B, Papilloma virus, Malaria, Tuberculosis, Herpes Simplex Virus, Chlamydia, and Influenza, or a biological component thereof, for example, a viral or bacterial polypeptide. In embodiments of the invention the adjuvant polypeptide is covalently linked to the antigen. For example, the Man fusion protein is linked to the antigen via a covalent bond such as a peptide bond. The antigen is fused to the N-terminus or C-terminus of the mucin polypeptide. Alternatively, the antigen is fused to an internal amino acid of the mucin polypeptide. By “internal amino acid” is meant an amino acid that is not at the N-terminal or C-terminal of a polypeptide. Similarly, the antigen is operably linked to the second polypeptide of the adjuvant polypeptide, most typically via a covalent bond such as a peptide bond. The antigen is fused to the N-terminus or C-terminus of the second polypeptide of the adjuvant polypeptide. Alternatively, the antigen is fused to an internal amino acid of the second polypeptide of the adjuvant polypeptide.

The Man fusion proteins includes a heterologous signal sequence (i.e., a polypeptide sequence that is not present in a polypeptide encoded by a mucin or a globulin nucleic acid) at its N-terminus. For example, the native mucin or alpha-glycoprotein signal sequence can be removed and replaced with a signal sequence from another protein. In certain host cells (e.g., mammalian host cells), expression and/or secretion of polypeptide can be increased through use of a heterologous signal sequence.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. The fusion gene is synthesized by conventional techniques including automated DNA synthesizers. Alternatively, PCR amplification of gene fragments is carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that encode a fusion moiety (e.g., an F_{c} region of an immunoglobulin heavy chain). A mucin or an alpha-globulin encoding nucleic acid can be cloned into such an expression vector such that the fusion moiety is linked in-frame to the immunoglobulin protein.

Man fusion polypeptides may exist as oligomers, such as dimers, trimers or pentamers. Preferably, the Man fusion polypeptide is a dimer.

The first polypeptide, and/or nucleic acids encoding the first polypeptide, is constructed using mucin/mucin-type or alpha-globulin encoding sequences known in the art. Suitable sources for mucin polypeptides and nucleic acids encoding mucin polypeptides include GenBank Accession Nos. NP663625 and NM145650, CAD10625 and AJ417815, XP140694 and XM140694, XP006867 and XM006867 and NP00331777 and NM009151 respectively, and are incorporated herein by reference in their entirety. Suitable sources for alpha-globulin polypeptides and nucleic acids encoding alpha-globulin polypeptides include GenBank Accession Nos. AAH26238 and BC026238; NP000598; and BC012725, AAH12725 and BC012725, and NP44570 and NM053288 respectively, and are incorporated herein by reference in their entirety.

The mucin polypeptide moiety is provided as a variant mucin polypeptide having a mutation in the naturally-occurring mucin sequence (wild type) that results in increased carbohydrate content (relative to the non-mutated sequence). For example, the variant mucin polypeptide comprised additional O-linked glycosylation sites compared to the wild-type mucin. Alternatively, the variant mucin polypeptide comprises an amino acid sequence mutations that results in an increased number of serine, threonine or proline residues as compared to a wild type mucin polypeptide. This increased carbohydrate content can be assessed by determining the protein to carbohydrate ratio of the mucin by methods known to those skilled in the art.

Similarly, the alpha-globulin polypeptide moiety is provided as a variant alpha-globulin polypeptide having a mutation in the naturally-occurring alpha-globulin sequence (wild type) that results in increased carbohydrate content (relative to the non-mutated sequence). For example, the variant alpha-globulin polypeptide comprised additional N-linked glycosylation sites compared to the wild-type alpha-globulin.

Alternatively, the mucin or alpha-globulin polypeptide moiety is provided as a variant mucin or alpha-globulin polypeptide having mutations in the naturally-occurring mucin or alpha-globulin sequence (wild type) that results in a mucin or alpha-globulin sequence more resistant to proteolysis (relative to the non-mutated sequence).

The first polypeptide includes full-length PSGL-1. Alternatively, the first polypeptide comprise less than full-length PSGL-1 polypeptide such as the extracellular portion of PSGL-1. For example the first polypeptide less than 400 amino acids in length, e.g., less than or equal to 300, 250, 150, 100, 50, or 25 amino acids in length.

The first polypeptide includes full-length alpha acid-globulin. Alternatively, the first polypeptide comprises less than full-length alpha acid globulin polypeptides. For example the first polypeptide less than 200 amino acids in length, e.g., less than or equal to 150, 100, 50, or 25 amino acids in length.

The second polypeptide is preferably soluble. In some embodiments, the second polypeptide includes a sequence that facilitates association of the Man fusion polypeptide with a second mucin or alpha globulin polypeptide. The second polypeptide includes at least a region of an immunoglobulin polypeptide. “At least a region” is meant to include any portion of an immunoglobulin molecule, such as the light chain, heavy chain, F_{c} region, F_{ab} region, Fᵥ region or any fragment thereof. Immunoglobulin fusion polypeptide are known in the art and are described in e.g., U.S. Pat. Nos. 5,516,964; 5,225,538; 5,428,130; 5,514,582; 5,714,147; and 5,455,165.

The second polypeptide comprises a full-length immunoglobulin polypeptide. Alternatively, the second polypeptide comprises less than full-length immunoglobulin polypeptide, e.g., a heavy chain, light chain, Fab, Fab₂, Fv, or Fc. Preferably, the second polypeptide includes the heavy chain of an immunoglobulin polypeptide. More preferably the second polypeptide includes the F_{c} region of an immunoglobulin polypeptide.

The second polypeptide has less effector function than the effector function of an F_{c} region of a wild-type immunoglobulin heavy chain. Alternatively, the second polypeptide has similar or greater effector function than that of an F_{c} region of a wild-type immunoglobulin heavy chain. An F_{c} effector function includes for example, F_{c} receptor binding, complement fixation and T cell depleting activity (see for example, U.S. Pat. No. 6,136,310). Methods of assaying T cell depleting activity, F_{c} effector function, and antibody stability are known in the art. In one embodiment the second polypeptide has low or no affinity for the F_{c} receptor. Alternatively, the second polypeptide has low or no affinity for complement protein C1q.

Another aspect of the invention pertains to vectors, preferably expression vectors, containing a nucleic acid encoding mucin polypeptides, or derivatives, fragments, analogs or homologs thereof. The vector contains a nucleic acid encoding a mucin or alpha globulin polypeptide operably linked to a nucleic acid encoding an immunoglobulin polypeptide, or derivatives, fragments analogs or homologs thereof. Additionally, the vector comprises a nucleic acid encoding a glycotransferase. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., Man fusion polypeptides, mutant forms of Man fusion polypeptides, etc.).

The recombinant expression vectors of the invention can be designed for expression of Man fusion polypeptides in prokaryotic or eukaryotic cells. Preferably the Man fusion proteins are expressed in eukatyotic cells. Most preferably, the Man-fusion proteins are expressed in a yeast cell such as Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichiapyperi, Pichia stiptis, Pichia methanolica, Pichia sp., Saccharomyces cerevisiae, Saccharomyces sp., Hansenulapolymorpha, Kluyveromyces sp., Candida albicans, Aspergillus nidulans, or Trichoderma reesei.

The Man fusion polypeptide expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRy88 (Schultz et al., 1987. Gene 54: 113-123), pYES2 (Invitrogen Corporation, San Diego, Calif.), and picZ (InVitrogen Corp, San Diego, Calif.).

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, Man fusion polypeptides can be expressed in bacterial cells such as E. coli, insect cells, yeast or mammalian cells (such as human, Chinese hamster ovary cells (CHO) or COS cells). Other suitable host cells are known to those skilled in the art. Preferably, the Man fusion polypeptides are expressed in a lower eukaryotic cell. Preferably, the host cell is yeast.

Lower eukaryotic cells possess several advantages over the use of mammalian cells for large scale industrial production of recombinant proteins. The ability to grow to high cell densities in combination with an efficient secretion system for recombinant proteins allows for high extracellular productivity and simplified downstream processing [37]. In a particular embodiment, the host cell is P. pastoris. P. pastoris is a robust fermentation organism with a preference for respiratory growth which makes P. pastoris comparably insensitive to oxygen limitation zones common in large bioreactors [38]. In contrast, fermentative organisms like Escherichia coli generate fermentative by-products under oxygen limitation such as acetate and ethanol which have shown to be of major concern during scale up with such organisms [39]. Compared to mammalian cell lines like CHO (Chinese Hamster ovary) cells, genetic manipulation, selection and cultivation are much faster with P. pastoris and are likely to significantly reduce production economics for biopharmaceuticals expressed in this host. The absence of viral inclusions pathogenic for humans and high product homogeneity of certain recombinant glycoproteins derived from P. pastoris simplifies regulatory considerations [40, 41].

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the fusion polypeptides or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) Man fusion polypeptides. Accordingly, the invention further provides methods for producing Man fusion polypeptides using the host cells of the invention. In one embodiment, the method comprises culturing the host cell of invention (into which a recombinant expression vector encoding Man fusion polypeptides has been introduced) in a suitable medium under suitable conditions such that Man fusion polypeptides are produced. Preferably, the host cell is a lower eukaryotic cell, such as a yeast cell. In a particular embodiment, the Man fusion polypeptides are produced by the host cell using fed-batch fermentation. The host cell may be cultured in a conventional shake flask or a bioreactor. In one embodiment, the host cell is cultured under conditions which permit cell growth (i.e., growth phase) until a suitable/desired cell density is reached. A suitable/desired cell density may be 10%, 25%, 30%, 35%, 40%, 45%, 50%, 55%, 60%, 65%, 70%, 75%, 80%, 85%, 90%, 95% or 100% confluency (i.e., coverage or proliferation that the cells are allowed over or throughout the culture medium). For example, a cell density of approximately OD₆₀₀ 5-6 or 1 g DCW/I to approximately 500 g DCWI, (e.g., 10 g DCW/I to 350 g DCW/I, 25 g DCW/I to 250 g DCW/I, or 50 g DCW/I to 150 g DCW/I) is reached. Suitable culture conditions which permit cell growth may comprise a neutral or near neutral pH (e.g., pH 6.0 to 8.0) and 29° C. to 37° C.

Once the cells reach peak density, the cells are induced (i.e., induction phase) to produce the Man fusion polypeptide of the invention under conditions which permit maintenance and or/continued growth of the cell culture. Suitable inducers will depend on the type of fed-batch fermentation technique employed and are known to one of ordinary skill in the art. The cell culture conditions during the growth phase may be maintained during the induction phase. Preferably, the cell culture conditions during the induction phase are adjusted from the cell culture conditions during the growth phase to permit decreased fragmentation and increased glycosylation of the Man fusion polypeptide produced by said cells. Suitable conditions which permit decreased fragmentation and increased glycosylation of the recombinant proteins secreted by the host cells comprise lowering the pH from a neutral or near neutral pH during the growth phase (e.g., pH 6.0 to 8.0) to create a more acidic environment during the induction phase. For example, the pH is lowered from a neutral or near neutral pH during the growth phase (e.g., pH 6.0 to 8.0) to a more acidic pH. Suitable acidic pH conditions during the induction phase include pH 5.0, 4.5, 4.0, 3.9, 3.8, 3.7, 3.6, 3.5, 3.4, 3.3, 3.2, 3.1, 3.0, 2.5, 2.0 and 1.0. Surprisingly, an acidic environment during the induction phase does not contribute to increased degradation of the secreted proteins. For example, lowering the pH from 6.0 during the growth phase to pH 3.5 during the induction phase results in less degradation of the secreted protein and increased O-linked glycosylation sites on the Man fusion polypeptide of the invention as compared to the corresponding wild-type protein (e.g., wild type mucin or alpha glycoprotein). In another embodiment, the method further comprises isolating Man polypeptide from the medium or the host cell using conventional techniques known to one of ordinary skill in the art.

The Man fusion polypeptides may be isolated and purified in accordance with conventional conditions, such as extraction, precipitation, chromatography, affinity chromatography, electrophoresis or the like. For example, the immunoglobulin fusion proteins may be purified by passing a solution through a column which contains immobilized protein A or protein G which selectively binds the F_{c} portion of the fusion protein. See, for example, Reis, K. J., et al., J. Immunol. 132:3098-3102 (1984); PCT Application, Publication No. WO87/00329. The fusion polypeptide may then be eluted by treatment with a chaotropic salt or by elution with aqueous acetic acid (1 M).

Alternatively, Man fusion polypeptides according to the invention can be chemically synthesized using methods known in the art. A variety of protein synthesis methods are common in the art, including synthesis using a peptide synthesizer. See, e.g., Peptide Chemistry, A Practical Textbook, Bodasnsky, Ed. Springer-Verlag, 1988; Merrifield, Science 232: 241-247 (1986); Barany, et al, Intl. J. Peptide Protein Res. 30: 705-739 (1987); Kent, Ann. Rev. Biochem. 57:957-989 (1988), and Kaiser, et al, Science 243: 187-198 (1989). The polypeptides are purified so that they are substantially free of chemical precursors or other chemicals using standard peptide purification techniques. The language “substantially free of chemical precursors or other chemicals” includes preparations of peptide in which the peptide is separated from chemical precursors or other chemicals that are involved in the synthesis of the peptide. In one embodiment, the language “substantially free of chemical precursors or other chemicals” includes preparations of peptide having less than about 30% (by dry weight) of chemical precursors or non-peptide chemicals, more preferably less than about 20% chemical precursors or non-peptide chemicals, still more preferably less than about 10% chemical precursors or non-peptide chemicals, and most preferably less than about 5% chemical precursors or non-peptide chemicals.

Chemical synthesis of polypeptides facilitates the incorporation of modified or unnatural amino acids, including D-amino acids and other small organic molecules. Replacement of one or more L-amino acids in a peptide with the corresponding D-amino acid isoforms can be used to increase the resistance of peptides to enzymatic hydrolysis, and to enhance one or more properties of biologically active peptides, i.e., receptor binding, functional potency or duration of action. See, e.g., Doherty, et al., 1993. J. Med. Chem. 36: 2585-2594; Kirby, et al., 1993. J. Med. Chem. 36:3802-3808; Morita, et al., 1994. FEBS Lett. 353: 84-88; Wang, et al., 1993. Int. J. Pept. Protein Res. 42: 392-399; Fauchere and Thiunieau, 1992. Adv. Drug Res. 23: 127-159.

Introduction of covalent cross-links into a peptide sequence can conformationally and topographically constrain the polypeptide backbone. This strategy can be used to develop peptide analogs of the fusion polypeptides with increased potency, selectivity and stability. Because the conformational entropy of a cyclic peptide is lower than its linear counterpart, adoption of a specific conformation may occur with a smaller decrease in entropy for a cyclic analog than for an acyclic analog, thereby making the free energy for binding more favorable. Macrocyclization is often accomplished by forming an amide bond between the peptide N- and C-termini, between a side chain and the N- or C-terminus [e.g., with K₃Fe(CN)₆ at pH 8.5] (Samson et al., Endocrinology, 137: 5182-5185 (1996)), or between two amino acid side chains. See, e.g., DeGrado, Adv Protein Chem, 39: 51-124 (1988). Disulfide bridges are also introduced into linear sequences to reduce their flexibility. See, e.g., Rose, et al., Adv Protein Chem, 37: 1-109 (1985); Mosberg et al., Biochem Biophys Res Commun, 106: 505-512 (1982). Furthermore, the replacement of cysteine residues with penicillamine (Pen, 3-mercapto-(D) valine) has been used to increase the selectivity of some opioid-receptor interactions. Lipkowski and Carr, Peptides: Synthesis, Structures, and Applications, Gutte, ed., Academic Press pp. 287-320 (1995).

### Methods of Immunization

The Man-fusion proteins of the invention are also useful as vaccine adjuvant. The vaccines of the present invention have superior immunoprotective and immunotherapeutic properties over other vaccine lacking adjuvant polypeptides. Mucin-Ig fusion protein-containing vaccines have enhanced immunogenicity, safety, tolerability and efficacy. For example, the enhanced immunogenicity of the vaccine of the present invention may be greater than comparative non-adjuvant polypeptide-containing vaccines by 1.5-fold, 2-fold, 3-fold, 5-fold, 10-fold, 20-fold, 50-fold, 100-fold or more, as measured by stimuation of an immune response such as antibody production and/or secretion, activation and expansion of T cells, and cytokine expression (e.g., production of interleukins).

The cell surface of cancer cells often contains specific carbohydrates, polypeptides and other potential antibody epitopes that are not presence on the surface of non-cancerous cells. This antigen disparity allows the body's immune system to detect and respond to cancer cells. Mucin polypeptides have been associated with numerous cancers. For example, PSGL-1 has been associated with cancers, including lung cancer and acute myeloid leukemia (See Kappelmayer et al., Br J. Haematol. 2001, 115(4):903-9). Also, MUC1-specific antibodies have been detected in sera from breast, pancreatic and colon cancer patients. It is clear that mucins can be recognized by the human immune system; therefore, immunity against tumor cells expressing specific antigens will be induced by vaccines containing mucin-Ig fusion proteins and a tumor cell-specific antigen. Immunity to tumor cells is measured by the extent of decrease of tumor size, decreased tumor vascularization, increased subject survival, or increased tumor cell apoptosis.

The invention provides a method of immunization of a subject. A subject is immunized by administration to the subject the vaccine including an adjuvant polypeptide, e.g. a Man fusion protein and an antigen. The subject is at risk of developing or suffering from an infection, e.g., bacterial, viral or fungal. Infections include, Hepatitis C, HIV, Hepatitis B, Papilloma virus, Malaria, Tuberculosis, Herpes Simplex Virus, Chlamydia, or Influenza. Alternatively, the subject is at risk of developing or suffering from cancer. The cancer is for example breast, lung, colon, prostate, pancreatic, cervical cancer or melanoma.

The methods described herein lead to a reduction in the severity or the alleviation of one or more symptoms of a infection or cancer. Infection and cancers diagnosed and or monitored, typically by a physician using standard methodologies. A subject requiring immunization is identified by methods know in the art. For example subjects are immunized as outlined in the CDC's General Recommendation on Immunization (51(RR02) pp 1-36). Cancer is diagnosed for example by physical exam, biopsy, blood test, or x-ray.

The subject is e.g., any mammal, e.g., a human, a primate, mouse, rat, dog, cat, cow, horse, pig. The treatment is administered prior to diagnosis of the disorder. Alternatively, treatment is administered after diagnosis.

Efficaciousness of treatment is determined in association with any known method for diagnosing or treating the particular disorder. Alleviation of one or more symptoms of the disorder indicates that the compound confers a clinical benefit. By “efficacious” is meant that the treatment leads to decrease in size, prevalence, or metastatic potential of the cancer in a subject. When treatment is applied prophylactically, “efficacious” means that the treatment retards or prevents a tumor from forming or retards, prevents, or alleviates a symptom of the cancer. Assessment of cancer is made using standard clinical protocols. Similarly, increased immunization clinical benefit is determined for example by decreased physician visits, and decreased disease burden in the community.

### Methods of Increasing Antibody Secretion

The invention provides a method of increasing or stimulating production and/or secretion of antibodies in a cell. The cell is an antibody forming cell such as a B-cell. Alternatively, the cell is a cell that augments antibody production by a B cell such as a T-cell (Th and Tc), macrophage, dendritic cell

Antibody secretion by a cell is increased by contacting the cell with the vaccine including an adjuvant polypeptide and an antigen. Antibody secretion by a cell can be increased directly, such as by stimulating B cells, or indirectly, such as by stimulating T cells (e.g., helper T cells), which activated T cells then stimulate B cells. Increased antibody production and/or secretion is measured by methods known to those of ordinary skill in the art, including ELISA, the precipitin reaction, and agglutination reactions.

### Methods of Increasing Immune Cell Activation

The invention provides a method of activating or stimulating an immune cell (e.g., a B cell or a T cell). T cell activation is defined by an increase in calcium mediated intracellular cGMP, or an increase in cell surface receptors for IL-2. For example, an increase in T cell activation is characterized by an increase of calcium mediated intracellular cGMP and or IL-2 receptors following contacting the T cell with the vaccine, compared to in the absence of the vaccine. Intracellular cGMP is measured, for example, by a competitive immunoassay or scintillation proximity assay using commercially available test kits. Cell surface IL-2 receptors are measured, for example, by determining binding to an IL-2 receptor antibody such as the PC61 antibody. Immune cell activation can also be determined by measuring B cell proliferative activity, polyclonal immunoglobulin (Ig) production, and antigen-specific antibody formation by methods known in the art.

### Pharmaceutical Compositions

The fusion peptides of the invention can be formulated in pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal or patch routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Whether it is a polypeptide, peptide, or nucleic acid molecule, other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in REMINGTON'S PHARMACEUTICAL SCIENCES, 16th edition, Osol, A. (ed), 1980.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they could be produced in the target cells by expression from an encoding gene introduced into the cells, e.g. in a viral vector (a variant of the VDEPT technique—see below). The vector could be targeted to the specific cells to be treated, or it could contain regulatory elements, which are switched on more or less selectively by the target cells.

Alternatively, the agent could be administered in a precursor form, for conversion to the active form by an activating agent produced in, or targeted to, the cells to be treated. This type of approach is sometimes known as ADEPT or VDEPT; the former involving targeting the activating agent to the cells by conjugation to a cell-specific antibody, while the latter involves producing the activating agent, e.g. a vaccine or fusion protein, in a vector by expression from encoding DNA in a viral vector (see for example, EP-A-415731 and WO 90/07936).

In a specific embodiment of the present invention, nucleic acids include a sequence that encodes a vaccine, or functional derivatives thereof, are administered to modulate immune cell activation by way of gene therapy. In more specific embodiments, a nucleic acid or nucleic acids encoding a vaccine or fusion protein, or functional derivatives thereof, are administered by way of gene therapy. Gene therapy refers to therapy that is performed by the administration of a specific nucleic acid to a subject. In this embodiment of the present invention, the nucleic acid produces its encoded peptide(s), which then serve to exert a therapeutic effect by modulating function of the disease or disorder. Any of the methodologies relating to gene therapy available within the art may be used in the practice of the present invention. See e.g., Goldspiel, et al., 1993. Clin Pharm 12: 488-505.

In a preferred embodiment, the therapeutic comprises a nucleic acid that is part of an expression vector expressing any one or more of the vaccines, fusion proteins, or fragments, derivatives or analogs thereof, within a suitable host. In a specific embodiment, such a nucleic acid possesses a promoter that is operably-linked to coding region(s) of a fusion protein. The promoter may be inducible or constitutive, and, optionally, tissue-specific. In another specific embodiment, a nucleic acid molecule is used in which coding sequences (and any other desired sequences) are flanked by regions that promote homologous recombination at a desired site within the genome, thus providing for intra-chromosomal expression of nucleic acids. See e.g., Koller and Smithies, 1989. Proc Natl Acad Sci USA 86: 8932-8935.

Delivery of the Therapeutic nucleic acid into a patient may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. In a specific embodiment of the present invention, a nucleic acid is directly administered in vivo, where it is expressed to produce the encoded product. This may be accomplished by any of numerous methods known in the art including, e.g., constructing the nucleic acid as part of an appropriate nucleic acid expression vector and administering the same in a manner such that it becomes intracellular (e.g., by infection using a defective or attenuated retroviral or other viral vector; see U.S. Pat. No. 4,980,286); directly injecting naked DNA; using microparticle bombardment (e.g., a “Gene Gun®; Biolistic, DuPont); coating the nucleic acids with lipids; using associated cell-surface receptors/transfecting agents; encapsulating in liposomes, microparticles, or microcapsules; administering it in linkage to a peptide that is known to enter the nucleus; or by administering it in linkage to a ligand predisposed to receptor-mediated endocytosis (see, e.g., Wu and Wu, 1987. J Biol Chem 262: 4429-4432), which can be used to “target” cell types that specifically express the receptors of interest, etc.

An additional approach to gene therapy in the practice of the present invention involves transferring a gene into cells in in vitro tissue culture by such methods as electroporation, lipofection, calcium phosphate-mediated transfection, viral infection, or the like. Generally, the method of transfer includes the concomitant transfer of a selectable marker to the cells. The cells are then placed under selection pressure (e.g., antibiotic resistance) so as to facilitate the isolation of those cells that have taken up, and are expressing, the transferred gene. Those cells are then delivered to a patient. In a specific embodiment, prior to the in vivo administration of the resulting recombinant cell, the nucleic acid is introduced into a cell by any method known within the art including, e.g., transfection, electroporation, microinjection, infection with a viral or bacteriophage vector containing the nucleic acid sequences of interest, cell fusion, chromosome-mediated gene transfer, microcell-mediated gene transfer, spheroplast fusion, and similar methodologies that ensure that the necessary developmental and physiological functions of the recipient cells are not disrupted by the transfer. See e.g., Loeffler and Behr, 1993. Meth Enzymol 217: 599-618. The chosen technique should provide for the stable transfer of the nucleic acid to the cell, such that the nucleic acid is expressible by the cell. Preferably, the transferred nucleic acid is heritable and expressible by the cell progeny.

In preferred embodiments of the present invention, the resulting recombinant cells may be delivered to a patient by various methods known within the art including, e.g., injection of epithelial cells (e.g., subcutaneously), application of recombinant skin cells as a skin graft onto the patient, and intravenous injection of recombinant blood cells (e.g., hematopoietic stem or progenitor cells). The total amount of cells that are envisioned for use depend upon the desired effect, patient state, and the like, and may be determined by one skilled within the art.

Cells into which a nucleic acid can be introduced for purposes of gene therapy encompass any desired, available cell type, and may be xenogeneic, heterogeneic, syngeneic, or autogeneic. Cell types include, but are not limited to, differentiated cells such as epithelial cells, endothelial cells, keratinocytes, fibroblasts, muscle cells, hepatocytes and blood cells, or various stem or progenitor cells, in particular embryonic heart muscle cells, liver stem cells (International Patent Publication WO 94/08598), neural stem cells (Stemple and Anderson, 1992, Cell 71: 973-985), hematopoietic stem or progenitor cells, e.g., as obtained from bone marrow, umbilical cord blood, peripheral blood, fetal liver, and the like. In a preferred embodiment, the cells utilized for gene therapy are autologous to the patient.

The vaccines of the present invention also include one or more adjuvant compounds. Adjuvant compounds are useful in that they enhance long term release of the vaccine by functioning as a depot. Long term exposure to the vaccine should increase the length of time the immune system is presented with the antigen for processing as well as the duration of the antibody response. The adjuvant compound also interacts with immune cells, e.g., by stimulating or modulating immune cells. Further, the adjuvant compound enhances macrophage phagocytosis after binding the vaccine as a particulate (a carrier/vehicle function).

Adjuvant compounds useful in the present invention include Complete Freund's Adjuvant (CFA); Incomplete Freund's Adjuvant (IFA); Montanide ISA (incomplete seppic adjuvant); Ribi Adjuvant System (RAS); TiterMax; Syntex Adjuvant Formulation (SAF); Aluminum Salt Adjuvants; Nitrocellulose-adsorbed antigen; Encapsulated or entrapped antigens; Immune-stimulating complexes (ISCOMs); and Gerbu^{R} adjuvant.

### EXAMPLES

### Example 1

### Expression of the Mucin-Type (PSGL-1/MIGG_{2B}) and αl₁-Acid Glycoprotein (AGP/MIGG_{2B}) Fusion Proteins in the Yeast Pichia Pastoris

PSGL-1/mIgG_{2b} carries mainly O-glycans whereas AGP/mIgG_{2b} is exclusively N-glycosylated. The cDNA sequence for a fusion protein comprised of the extracellular part of the mucin-like protein, P-selectin glycoprotein ligand-1, or the whole coding sequence except the translational stop for α₁-acid glycoprotein, and the F_{c} part of mouse IgG_{2b} was subcloned into an expression vector for P. pastoris as follows.

### Construction of Expression Plasmids

The cDNA encoding PSGL-1/mIgG_{2b} was PCR amplified from the PSGL-1/mIgG_{2b} expression plasmid [27] using 5′-CGC GGG AAT TCC AGC TGT GGG ACA CCT GGG-3′ and 5′-GCG GGA ATT CTC ATT TAC CCG GAG ACC GGG AGA TG-3′ as forward and reverse primers, respectively, and was ligated into the multiple cloning site of the pPICZα vector (Invitrogen, Carlsbad, Calif., USA) following EcoR1 digestion. The cDNA encoding AGP-1/mIgG_{2b} was PCR amplified from the AGP/mIgG_{2b} expression plasmid [28] using 5′-CGC GGG AAT TCC AGA TCC CAT TG-3′ and 5′-GCG GGG TAC CTC ATT TAC CCG GAG ACC GGG AGA TG-3′ as forward and reverse primers, respectively. The AGP-1/mIgG_{2b} fragment was digested by EcoR1 and Kpn1 and, likewise, subcloned into the multiple cloning site of the pPICZα vector (Invitrogen). The sequences were confirmed by DNA sequencing.

### Plasmid Integration and Selection of High Producing Clones

The vectors pPICZαA:PSGL-1/mIgG_{2b} and pPICZαA:AGP/mIgG_{2b} were amplified in E. coli XL-1 Blue using 25 μg/ml Zeocin™ as selective agent. Following purification the vectors were linearized by PmeI (New England BioLabs) and transformed into P. pastoris GS115 cells according to standard procedures (Easy Comp™, Invitrogen). Transformants of the Mut⁺ phenotype were subsequently identified by growing nine clones from each transformation on MDH agar (minimal dextrose medium+histidine: 1.34% yeast nitrogen base, 4×10⁻³% histidine, 4×10⁻⁵% biotin, 2% glucose, 1.5% agar) and on MMH agar (minimal methanol+histidine; same as MDH but with 0.5% methanol instead of glucose) using P. pastoris GS115/Mut^{s} and P. pastoris GS115/pPICZ/lacZ/Mut⁺ as negative and positive control respectively. To screen for high expressing clones, seven transformants of each transformation exhibiting the Mut⁺ phenotype were selected and inoculated in BMGY (buffered glycerol complex medium:1% yeast extract, 2% peptone, 1% v/v glycerol, 1.34% yeast nitrogen base, 100 mM potassium phosphate pH 6.0, 4×10⁻⁵% biotin) and grown for 24 hours at 29° C., at 180 rpm (Shake Incubator model 481, Thermo Electron Corporation, USA). This was followed by a 72 hours induction period in BMMY (buffered methanol-complex medium: 0.5% methanol, 1% yeast extract, 2% peptone, 1.34% yeast nitrogen base, 100 mM potassium phosphate pH 6.0, 4×10⁻⁵% biotin) at 29° C., 180 rpm. Cell culture supernatants were then harvested by centrifuging at 10,000 g 10 min at 4° C. and the concentration of PSGL-1/mIgG_{2b} and AGP-1/mIgG_{2b} in the supernatants was assessed by ELISA using a goat anti-mouse IgG(Fc) antibody as described below.

### Bioreactor Cultivation

An inoculum was prepared by inoculating 50 ml BMGY media in a 500 ml shake flask with P. pastoris GS115 Mut⁺ encoding either PSGL-1/mIgG_{2b} or AGP-1/mIgG_{2b}. The culture was incubated at 180 rpm (Shake Incubator model 481, Thermo electron cooperation, USA) 29° C. until OD₆₀₀ was approximately two. The bioreactor cultivations were conducted according to a methanol limited fed-batch strategy (MLFB) (Invitrogen Pichia Fermentation Process Guidelines Version B 053002) in 1 L bioreactors (Biobundle, Applikon, the Netherlands) with an initial volume of 820 ml BMGY supplemented with 4% w/v glycerol and 1 g/L histidine. The glycerol batch phase was conducted at 29° C., pH 6.0. The induction phase was performed at pH 6.0 and 3.5. To reduce pH to 3.5 for the induction phase, the pH controller was set to 3.5 during the glycerol fed phase and allowed to be lowered by the metabolic activity of the cells. The pH was maintained by automatic addition of 15% NH₄OH. During the glycerol batch phase the dissolved oxygen (DO) concentration, measured by a pO₂ electrode, was kept at 30% of oxygen saturation by keeping the agitation fixed at 700 rpm and varying the aeration and supply of pure oxygen as needed. The pO₂ electrode was calibrated before inoculation with oxygen saturation at 29° C., pH 6.0, one atmosphere, aeration of 0.75 L/min and an agitation of 700 rpm. After the initial glycerol was consumed, indicated by a DO value of 100%, the cells were fed with 50 ml of a 50% w/v glycerol including 12 ml PTM₁ (0.6% CuSO₄×5H₂O, 8×10⁻³% NaI, 0.3% MnSO₄×H₂O, 0.02% NaMoO₄×2H₂O, 2×10⁻³% Boric Acid, 0.05% CoCl₂, 2% ZnCl₂, 6.5% FeSO₄×7H₂O, 0.02% Biotin, 0.5% v/v H₂SO₄) salts per liter glycerol at a rate of 12.5 ml/h. The DO was maintained at 30%. Following a 10 minutes starvation period a 100% methanol feed with 12 ml PTM₁ salts per litre methanol was initiated and adjusted to keep the DO at 40%. Aeration and pure oxygen feed were adjusted as needed. Before induction, 5 ml of a 3.5% histidine solution was injected to the bioreactor.

### Shake Flask Cultivation

A single colony of P. pastoris GS 115 Mut⁺ encoding either PSGL-1/mIgG_{2b} or AGP-1/mIgG_{2b} was transferred to 200 ml sterile BMGY media in a 1 L, polycarbonate e-flask (Nalgene) and incubated in shake incubator (Model 481, Thermo Electron Corporation, USA) at 180 rpm, at 29° C. until OD600 was 5-6. The cells were then pelleted by centrifugation at 1500×g for five minutes and resuspended in BMMY media. The resuspended cells were distributed to five, 1 L, polycarbonate e-flasks (Nalgene) containing 200 ml, sterile BMMY to give a starting OD₆₀₀ of 1 and incubated on shake incubator (Model 481, Thermo Electron corporation, USA) at 180 rpm, at 29° C. for 72 hours. To maintain induction, 1 ml of pure methanol was added to each 200 ml culture every 24 hours. Cell culture supernatants were harvested by centrifuging at 10000×g for ten minutes at +4° C. and filtering with sterile, 1000 ml, 0.2 μm pore size, poly ether sulfone (PES) vacuum filter (Nalgene). The supernatants were finally treated with 1 ml protease inhibitor cocktail (Sigma P8215) per litre supernatant and stored at +4° C.

Purification of PSGL-1/mIgG_{2b} and AGP-1/mIgG_{2b} Fusion Proteins

The clarified supernatants were loaded onto a 1 ml HiTrap MabSelect SuRe column (GE Healthcare) pre-equilibrated with PBS. The column was washed with 10 column volumes (CV) of PBS, and elution of recombinant fusion protein was achieved using 5 CV of 0.1 M sodium citrate, pH 3.0. The eluted 1 ml fractions were each neutralised with 300 μl 1 M Tris-HCl, pH 9.0. As judged from the elution profile, selected fractions were pooled and dialyzed extensively (12-14 kD cut-off) against MilliQ water at 4° C. Typically, the pooled volume was 3-4 ml. After dialysis, the samples were frozen and lyophilized. Samples were then dissolved in 1 ml MilliQ water and kept frozen at −80° C.

### Quantification of Fusion Protein Using ELISA

The concentrations of recombinant fusion protein in supernatants and in purified fractions were determined by a two-antibody sandwich ELISA method as previously described [29]. Briefly, 96-well ELISA plates (Corning) were coated with an affinity-purified, polyclonal goat anti-mouse IgG F_{c} antibody (Sigma) at a concentration of 10 μg/ml. The plate was blocked with 1% BSA (bovine serum albumin) in PBS which was also used for dilution of fusion protein as well as the second antibody (peroxidase-conjugated, anti-mouse IgG(Fc) antibody; Sigma). All incubations lasted for 2 hours. Between and after incubations the plates were washed with PBS containing 0.5% (v/v) Tween 20. Bound peroxidase-conjugated antibody was visualized with 3,3′,5,5′-tetramethylbenzidine dihydrochloride (TMB, Sigma). The reaction was stopped by the addition of 2 M H₂SO₄ and the absorbance read at 450 nm. The fusion protein concentration was estimated using a dilution series of purified mouse IgG_{2b} (Serotec) in blocking buffer as an internal standard.

### PNGase F Treatment

After dialysis and lyophilisation, purified PSGL-1/mIgG_{2b} was dissolved in sodium phosphate buffer (0.1 M, pH 7.6) with 25 mM EDTA and 2% Triton-X 100, and was boiled for four minutes. 20 U of PNGase F (Roche, Basel, Switzerland) was added and the sample incubated at 37° C. overnight. As a control, an equal volume of buffer was added instead of PNGase F.

### SDS-PAGE and Western Blotting

The recombinant proteins were analyzed by SDS-PAGE and Western blotting under non-reducing conditions using 4-12% gradient gels and MES buffer (Invitrogen). Protein gels were stained using the Pro Q Emerald 300 Glycoprotein detection kit in combination with Ruby (Molecular Probes). Western blot membranes were probed with biotinylated Concanavalin A (Con A; 10 μg/ml; Vector, Burlingame, Calif., USA), a mouse anti-PSGL-1 antibody (clone KPL-1, BD PharMingen, San Diego, Calif., USA) diluted 1:500, and an anti-orosomucoid (Alpha-1-Acid Glycoprotein) (DakoCytomation, Denmark) diluted 1:50. Secondary antibodies were peroxidase-conjugated goat anti-mouse IgG F(ab)′₂ (Sigma) diluted 1:50,000 and goat anti-rabbit IgG(H+L) (Sigma) 1:10,000. Peroxidase-conjugated Avidin D (Vector) 0.2 μg/ml was used to detect Con A binding. Bound lectins and antibodies were visualized by chemiluminescence using the ECL kit according to the manufacturer's instructions (Amersham Biosciences).

Chemical Release and Permethylation of O-Linked Glycans from Purified PSGL-1/mIgG_{2b}

Oligosaccharides were released by β-elimination as described [30]. Released oligosaccharides were evaporated under a stream of nitrogen at 45° C., and permethylated according to [31] with slight modifications as described [32].

### Mass Spectrometry Analyses

Released saccharides were characterized by mass spectrometry. Given the goal of engineering P. pastoris into synthesizing more human-like O-glycans, the focus of the structural prior to the instant invention.

Electrospray ionization-mass spectrometry (ESI-MS) in positive-ion mode was performed using an LCQ ion-trap mass spectrometer (ThermoFinnigan, San Jose, Calif.). The sample was dissolved in methanol:water (1:1) and introduced into the mass spectrometer at a flow rate of 50 μl/min. Nitrogen was used as sheath gas and the needle voltage set to 4.0 kV. The temperature of the heated capillary was set to 200° C.

### Results

PSGL-1/mIgG_{2b} and AGP-1/mIgG_{2b} Expression

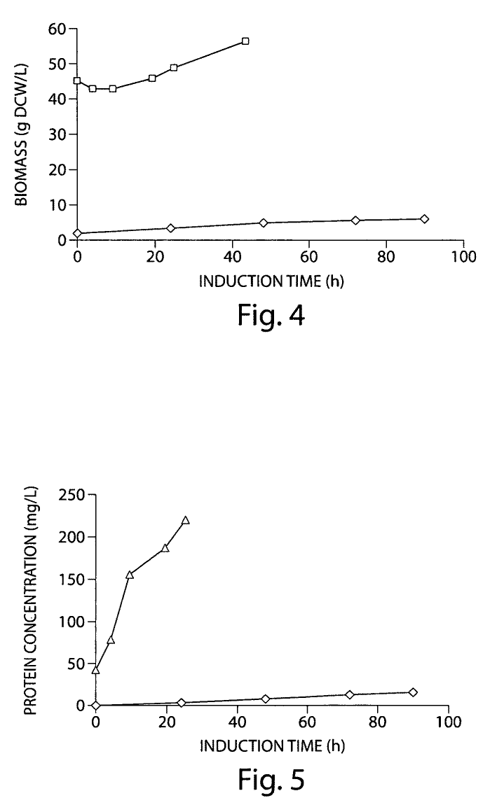

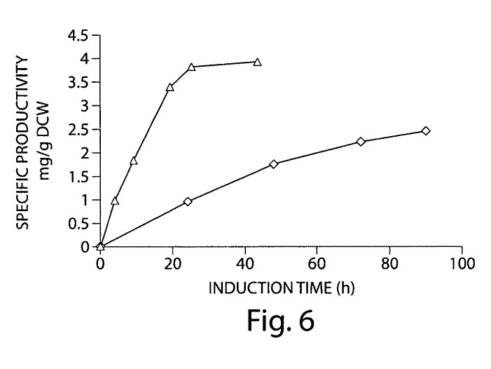

Expressions were conducted in bioreactor at pH 6.0 and 3.5, and in shake flasks at pH 6.0. Bioreactor cultivation naturally was supreme over shake flask cultivation as far as cell growth and product yield was concerned. Biomass prior to induction in the shake flask cultures started at 2 g/l (dry cell weight, DCW) and typically reached a cell mass of 5-6 g/l after 80-90 hours of induction. Cell mass after glycerol batch and fed batch phases in the bioreactor cultivation typically reached 45 g/l and continued to 50-60 g/l after 40-60 hours of induction depending on the methanol feeding rate (FIG. 4). pH did not appear to influence growth negatively under the conditions tested. Fusion protein concentration in culture supernatant from bioreactor cultivations ranged between 45-200 mg/l after 48 h induction with no apparent influence of pH. For shake flask cultures total productivities typically reached 10-15 mg/l after 72 h induction (FIG. 5). The maximum specific productivity for PSGL-1/mIgG_{2b} was 4 mg/g DCW for expression in bioreactor, and about 2.5 mg/g DCW for shake flask cultivation. Expression of AGP-1/mIgG_{2b} was lower at all times, with total productivities reaching 21 mg/l (specific productivity 0.5 mg/g DCW) in bioreactor cultivations and about 3.5 mg/l (specific productivity 0.7 mg/g DCW) in shake flask expressions (FIG. 6).

Purification of Fusion Protein using Protein A Chromatography

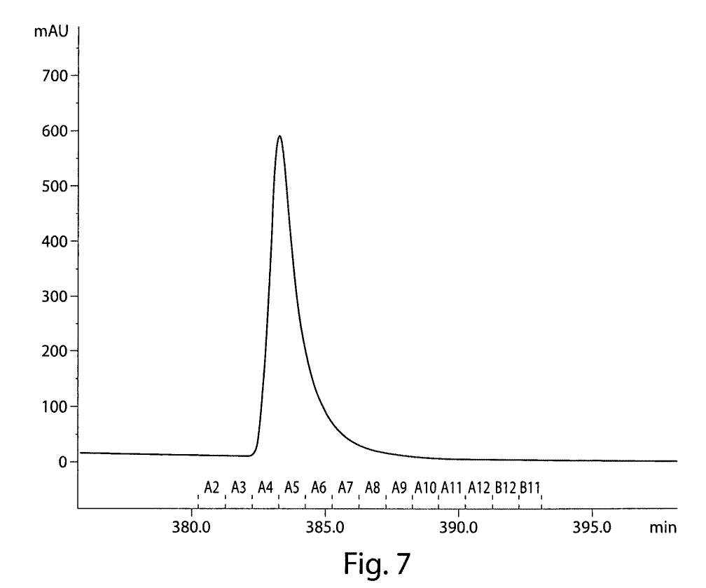

To save time in the purification process, different batches of PSGL-1/mIgG_{2b} were pooled before chromatography on the basis of their similarity on SDS-PAGE and Western blots, i.e. their different degradation and glycosylation patterns. In generic antibody purification strategy, the antibody is first captured by a Protein A chromatography step. Since the fusion proteins PSGL-1/mIgG_{2b} and AGP-1/mIgG_{2b} have the same ability to bind to a Protein A resin via its C-terminal F_{c} fragment, it was decided to try this strategy for both proteins. Here the fusion protein was captured from the culture supernatants by Protein A resin, washed to remove impurities and then eluted by lowering the pH. For all batches the elution profile looked good, with a single, quite symmetrical peak in the UV280 trace (FIG. 7). To protect the fusion protein and its carbohydrates, the eluted fractions were neutralized with Tris-HCl.

### SDS-PAGE and Western Blotting

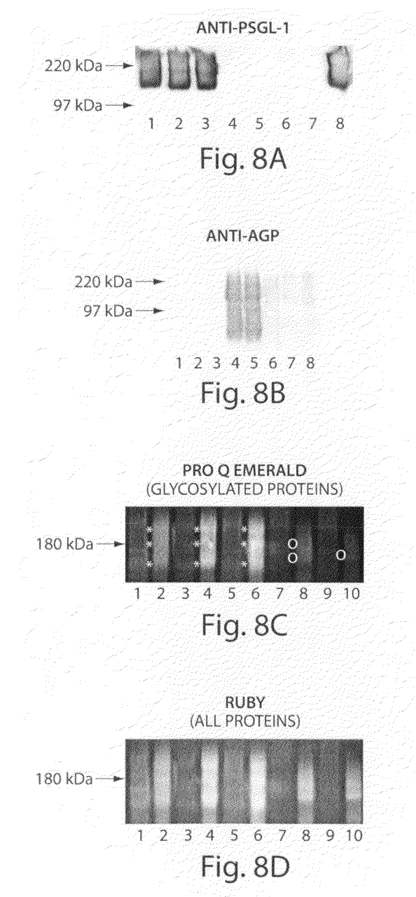

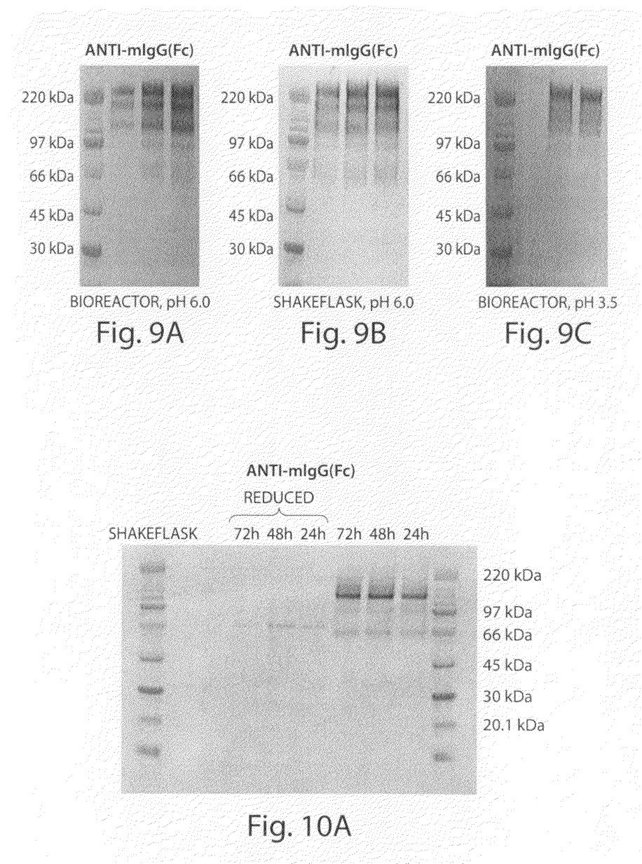

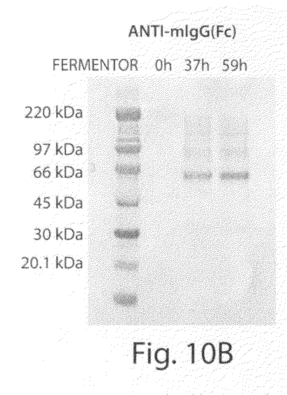

Western blot of PSGL-1/mIgG_{2b} indicated that P. pastoris produced an anti-PSGL-1- and anti-IgG(Fc) reactive protein of approximately 300 kDa under non-reducing conditions (FIG. 8A and FIG. 9). This is in accordance with previous observations [27, 33], where PSGL-1/mIgG_{2b} was produced mainly as a dimer. Additional bands of lower molecular weight were also found and were particularly pronounced for PSGL-1/mIgG_{2b} derived from bioreactor supernatants (compare FIGS. 9A and 9B). These bands most likely represent incompletely glycosylated forms of the fusion protein, monomers and possibly breakdown products. For the AGP-1/mIgG_{2b} expression, western blot analysis of bioreactor culture supernatants at pH 6.0 revealed an anti-IgG (F_{c})-reactive protein at approximately 66 kDa under non-reducing conditions, which is likely to represent the monomeric form of the dimeric fusion protein (FIG. 10B). Western analysis of reduced AGP-1/mIgG_{2b} derived from shakeflask supernatant revealed an anti-IgG (F_{c}) reactive protein slightly larger than 66 kDa. Reducing conditions should break the disulfide bond which keeps the two monomers together. Western blot of AGP-1/mIgG_{2b} derived from shake flask culture supernatants revealed an anti-AGP- and anti-IgG(Fc)-reactive protein at around 160 kDa under non-reducing conditions and most likely represents the dimeric form of AGP-1/mIgG_{2b} (FIG. 8B and FIG. 10A).

By expressing the recombinant proteins in the bioreactor at pH 3.5 less degradation was observed compared to expression at pH 6.0 (compare FIGS. 9A-C). For AGP/mIgG_{2b}, shake flask cultivation still displayed less degradation.

A glycoprotein staining kit (Pro Q Emerald) in combination with Ruby (all proteins) was used to detect glycosylated proteins in the cell culture supernatants and in the fractions obtained after affinity chromatography. As can be seen in FIG. 8C three bands are enriched after purification of each batch of cell supernatant (lanes 2, 4 and 6; marked with *).

### Bioreactor vs Shake Flask

Total productivity of the bioreactor cultivations were between 4 and 15 times higher than the shake flask cultivations and achieved in about ⅔ of the time. This is mainly related to the higher oxygen transfer rates achievable in the bioreactor allowing the fermentations to be run at much higher cell densities. The specific productivities were rather similar between the cultivation techniques, confirming the importance of reaching high cell densities before induction. P. pastoris can grow to very high cell densities in bioreactor cultures (130 g DCW/l) [42]. The cell densities reached during these fermentations were about 60 g DCW/l indicating that the total productivities could be increased further by increasing the cell densities. This should be done prior to induction however because during PSGL-1/mIgG_{2b} expression in bioreactor at pH 6.0 and 29° C., the cells stopped growing and producing after about 48 hours of induction which may indicate toxic effects of PSGL-1/mIgG_{2b} to the cells.

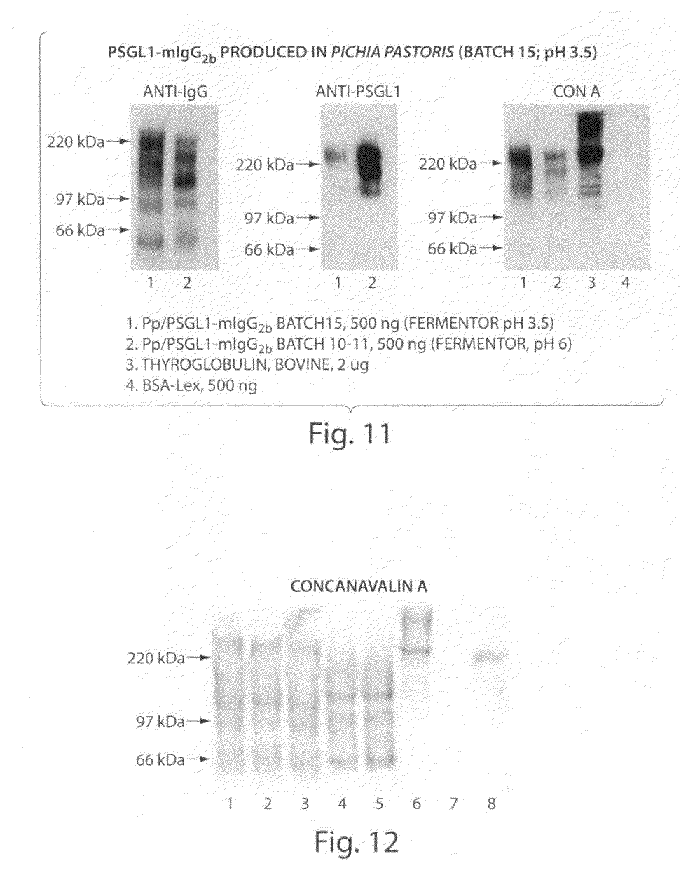

Under high expression rates secretion problems with the large PSGL-1/mIgG_{2b} could potentially result in the accumulation of PSGL-1/mIgG_{2b} inside the cells with detrimental effects to cell viability. Without intending to be bound by any theory, reducing the methanol- and pure oxygen feed to a lower induction pressure could solve such problems. Proteolytic degradation could limit the value of higher cell densities as indicated by the increased number of bands observed from western blots of bioreactor derived samples compared to samples derived from shake-flask cultures at pH 6.0. Reducing pH from 6.0 to 3.5 during the induction phase was shown to decrease the fragmentation of the recombinant proteins significantly as indicated by Western blot (compare FIGS. 9A and 9C). As Shown in FIG. 11, bioreactor derived samples at pH 3.5 instead of pH 6 gives less fusion protein break-down products and also a higher molecular weight of the dimeric fusion protein indicating a higher degree of glycosylation (compare lane 1 with lane 2; the stronger staining for lane 2 with anti-PSGL1 may be caused by less recognition of the fusion protein due to the higher glycosylation as is, apart from the higher MW in the anti-IgG figure, shown by the stronger staining by Con A to fusion protein produced at pH 3.5 as compared to pH 6).

Degradation of secreted recombinant proteins in high cell density cultures of P. pastoris is common and most likely occurs through the activity of secreted proteases, cell bound proteases or intracellular proteases released from lysed cells [43, 44]. Since P. pastoris secretes low levels of endogenous proteins and there are no documented extracellular proteases for this yeast, the majority of proteolytic activity in the culture medium is thought to be associated with non-specific and non-ATP requiring vacuolar proteases from lysed cells [37]. This is in accordance with the observation that AGP-1/mIgG_{2b} and PSGL-1/mIgG_{2b} were subjected to less degradation when expressed in shake flask cultures. The lower cell density and far less vigorous stirring in shake flasks are likely to reduce the number of dead cells and release of intracellular proteases. Furthermore, after the glycerol batch phase in shake flask cultures, the cells are pelleted and resuspended in fresh culture medium. This step washes away proteases potentially released during the initial glycerol phase. In contrast, the media is never changed during the bioreactor cultivations and proteases released during the glycerol batch phase may start degrade the recombinant proteins at the start of induction.

Western blot reveals that PSGL-1/mIgG_{2b} and AGP-1/mIgG_{2b} from the bioreactor cultivations are degraded from the start which is in accordance with this. Since the recombinant proteins were less degraded in acidic environments, serine proteases from lysed cells which have a higher pH optimum than for example aspartyl proteases, are likely to be the major cause of proteolytic degradation of PSGL-1/mIgG_{2b} and AGP-1/mIgG_{2b} during the fermentations at pH 6.0. Aspartyl proteases with lower pH optimum are hence not a problem for PSGL-1/mIgG_{2b}. AGP-1/mIgG_{2b} on the other hand still showed fragmentation at pH 3.5 and may be linked to the lower number of glycans compared to PSGL-1/mIgG_{2b}. The densely packed oligosaccharides of PSGL-1/mIgG_{2b} may, in part, function as a shield from proteolytic degradation.

The bands of various molecular weights observed on the Western blots may also come from differently glycosylated forms of the glycoproteins which is supported by the similar fragmentation patterns observed on the Western blots from bioreactor and shakeflask cultivation at pH 6.0 (compare FIGS. 9A and 9B). Alternatively, the glycans of the glycoproteins could be degraded by mannosidases in the culture media to a larger extent at pH 6.0 than at pH 3.5. The molecular weight of dimeric PSGL-1/mIgG_{2b} without its glycans is about 117 kDa. All bands observed at this molecular weight and above could thus potentially be the dimeric PSGL-1/mIG_{2b} with different glycosylation profiles. It has been suggested that weak bases such as amines would potentially affect glycosylation by diffusing into the cell in its neutral form and then accumulate in acidic compartments of the ER/golgi where they raise the pH and off-set the activity of pH sensitive enzymes, in turn influencing the glycosylation [45]. The base and nitrogen source used during the fermentations was ammonium hydroxide. At pH 6.0 a larger fraction of the ammonium ions is in its neutral form NH₃, than at pH 3.5. The neutral form could possibly diffuse through the cell wall easier than the charged form. Hence, the higher pH would potentially facilitate a greater accumulation of NH₄⁺ in acidic intracellular compartments of the golgi with aberrant glycosylation as a result. However, PSGL-1/mIgG_{2b} expressed in shake-flask cultivation at pH 6.0, where no ammonium hydroxide was used, still displayed a certain level of fragmentation with a pattern similar to that of samples from bioreactor expressions at pH 6.0. This suggests a different mechanism.

PSGL-1/mIgG_{2b} Produced in P. pastoris Carries Mannose Containing O-Glycans

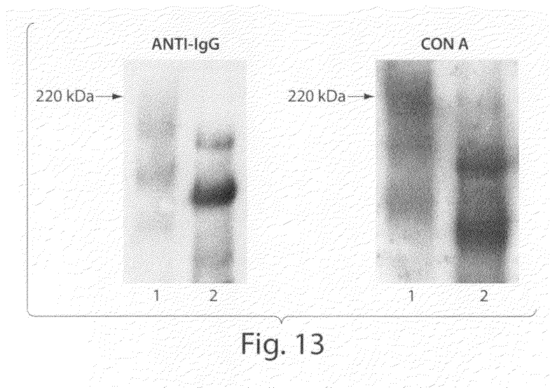

The lectin Con A was used in Western blotting analyses to investigate the presence of determinants containing mannose on the fusion protein produced in P. pastoris. Con A bound strongly to PSGL-1/mIgG_{2b} produced in P. pastoris. It also bound weakly to PSGL-1/mIgG_{2b} produced by CHO-PSLe^{x} cells (FIG. 12, lane 8). This is most likely due to the core mannoses in the complex type N-glycans present on the fusion protein. The binding to P. pastoris produced PSGL-1/mIgG_{2b} resisted PNGase F treatment (FIG. 13) indicating that the carbohydrate determinants recognized by the Con A lectin was carried also on O-glycans. The lectin also bound to bovine thyroglobulin (FIG. 12, lane 6), which is known to contain high-mannose type N-glycans, [34].

Mass Spectrometry of Permethylated Oligosaccharides Released from Purified, Recombinant PSGL-1/mIgG_{2b} Produced in Pichia pastoris

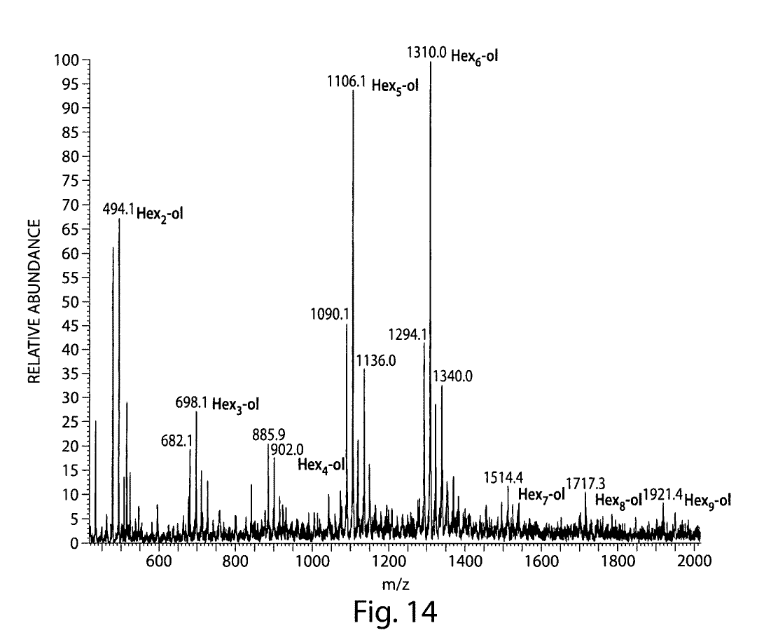

O-glycans released by β-elimination from 500 μg PSGL-1/mIgG_{2b} produced in P. pastoris were characterized using mass spectrometry following their permethylation. The sample was dissolved in methanol/water and ESI-MS carried out in the positive-ion mode with detection of [M+Na]⁺ ions. Five peaks corresponding to fragments with masses explained by the sodiated molecular ions of permethylated Hex₂₋₆ structures were seen (FIG. 14). These findings correspond well with O-glycans of P. pastoris derived glycoproteins characterized by Trimble et. al. [23]. In addition, three peaks corresponding to Hex₇₋₉ structures were seen. Hex₂₋₈ structures were confirmed by MSn analyses, while the peak most likely corresponding to Hex₉ were too small for MSn analysis.

### Example 2

### Assess the Ability of Pichia pastoris-Produced PSGL-1/MIGG_{2B} and AGP/MIGG_{2B} To Bind Macrophage Mannose Receptor, DC-Sign and Mannan Binding Lectin

Immunoglobulin fusion proteins of PSGL-1 and AGP produced in wild type Pichia and purified, as described in Example 1, were used in experiments to assess their ability to bind to recombinant human macrophage mannose receptor (MMR), DC-SIGN/F_{c} chimera and mannan binding lectin (MBL) Using Biacore (real time surface plasmon resonance spectroscopy) analysis as follows.

### Real Time Surface Plasmon Resonance Spectroscopy and Data Evaluation

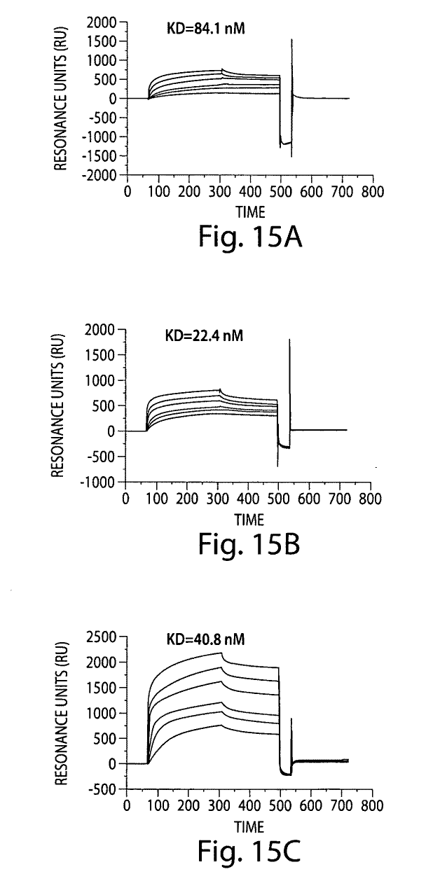

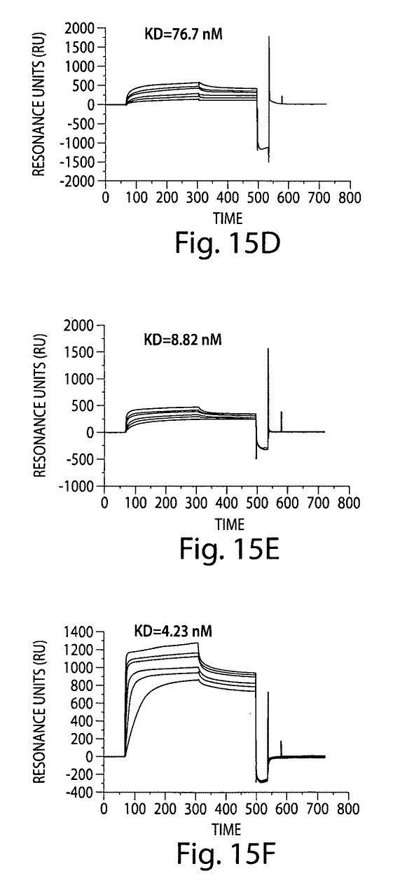

Analyses were performed using a Biacore 2000 instrument (Biacore, GE Healthcare, Uppsala, Sweden). Recombinant human macrophage mannose receptor (MMR), DC-SIGN/F_{c} chimera and mannan binding lectin (MBL) were purchased from R & D Systems and immobilized on a CM5 sensor chip using amine coupling chemistry according to the manufacturer's instructions. Briefly, activation of the surface was made with EDC/NHS 1:1, 10 μL/min for 7 minutes and the receptors were dissolved in sodium acetate buffer pH 4.5 at concentrations of 15 μg/ml for MMR, 10 μg/ml for DC-SIGN and 90 μg/ml for MBL and immobilized at 10 μl/min for 7 minutes. Deactivation of excess of reactive groups was made with ethanolamine, 20 μl/min for 7 minutes. The immobilization levels of the receptors were 13745 RU for MMR, 8312 RU for DC-SIGN and 5676 RU for MBL. The analytes AGP-1/mIgG_{2b} and PSGL-1/mIgG_{2b} were dissolved in HBS-P buffer with 1 mM CaCl₂ and 1 mM MgCl₂ and flown over the CM5 sensor chip with a rate of 20 μl/min for 4 minutes with 2.5 minute waiting time. Regeneration of the surfaces was achieved by injection of glycine pH 2.2 at 30 μl/min for 40 seconds. One channel on the CM5 sensor chip was immobilized only with buffer and was used as blank sensograms for subtraction of the bulk refractive index background. Data were calculated using BIAevaluation 4.1 software (Biacore, GE Healthcare) and the apparent equilibrium dissociation constants (Kd) were calculated by plotting steady state binding levels against the analyte concentrations for several concentrations simultaneously. The apparent K_{d} values are presented in FIG. 15. The immobilized surface of the sensor chip was tested with mannan to verify that the surface still was active after the regeneration procedure. D-mannose and oligo-mannose-9 were also tested for their binding to these receptors but no (D-mannose) or poor (oligo-mannose-9) binding was observed.

The apparent equilibrium dissociation constants, K_{d}, for AGP-1/mIgG_{2b} and PSGL-1/mIgG_{2b} ranged between 84.1 nM and 4.23 nM for all recombinant receptors, indicating a specific binding in all cases (FIG. 15). In contrast, no binding was observed for D-mannose and poor binding was observed for oligo-mannose-9. The dissociation constants for each case are listed in Table 1.

TABLE 1
Apparent dissociation constants (K_{d}): Biacore Analysis

| | MMR | DC-SIGN | MBL |
|---|---|---|---|
| PSGL-1/mlgG2b | 76.7 nM | 8.82 nM | 4.23 nM |
| AGP-1/mlgG2b | 84.1 nM | 22.4 nM | 40.8 nM |

The Biacore data indicates that MMR, DC-SIGN and MBL all bind PSGL-1/mIgG_{2b} and AGP-1/mIgG_{2b} with high affinity. Hence, both recombinant glycoproteins should have the potential to target these receptors in vivo and possibly promote enhanced immune responses. Free D-mannose did not bind to the receptors under the experimental conditions used whereas oligo-mannose-9 exhibited poor binding. Without intending to be bound by any theory, these results emphasize the fact that the high affinity binding observed may be achieved through multiple interactions between the carbohydrate epitope and the CRDs of the receptors which may include epitopes outside the principle sugar binding site.

Similar results for DC-SIGN supports this theory [35]. High affinity interactions between proteins and carbohydrates are typically achieved by the combined strength of many weak bonds from multiple binding sites—i.e. multivalence [25]. Both AGP-1/mIgG_{2b} and PSGL-1/mIgG_{2b} have the potential to carry multiple sugar chains which are likely to increase the binding strength to the receptors by targeting multiple CRDs of a receptor and/or secondary sites outside the primary sugar binding site. Many oligosaccharides of various sizes and types in close proximity could also form a clustered patch which would facilitate the high affinity binding observed from the Biacore analysis [36]. The specificity for certain carbohydrate epitopes and the arrangement of the CRDs of the receptors also influence the binding affinities.

### Receptor Structure and Carbohydrate Binding Specificity

The number and arrangement of the CRDs of MMR, DC-SIGN and MBL are quite different and are also likely to influence the binding affinities of the ligands. MMR has eight CRDs, located in the middle of the molecule, where at least three have been shown to be crucial for high affinity binding [11]. DC-SIGN has a single CRD at its terminal end and MBL has three CRDs at its terminal end [13, 17]. Thus, only MMR and MBL have the ability to mediate binding to several carbohydrate epitopes per molecule through their multiple CRDs. Based on this, it might be expected that MMR and MBL should bind with highest affinity to the glycoproteins due to multivalent binding. Surprisingly, MMR displays lowest affinities for the AGP-1/mIgG_{2b} and PSGL-1/mIgG_{2b} glycoproteins, contrary to what was expected. MMR also binds AGP-1/mIgG_{2b} and PSGL-1/mIgG_{2b} with similar affinities. Without intending to be bound by theory, this may be linked to the arrangement of the CRDs of MMR. Only DC-SIGN and MBL have their CRDs on the terminal end. In the Biacore experiments where surfaces are coated with a receptor, terminally located CRDs may have a better chance of reaching out to their ligands. In the case of MMR, the CRDs which are located in the middle of the molecule may be less available to bind their ligands. In vivo, however, where the receptors are more dispersed, the CRDs of MMR may be more accessible to its ligands and bind with higher affinity. This could be determined by immobilizing the recombinant glycoproteins and flow the receptors over them in the Biacore experiments. In this way, the CRDs of MMR would potentially be more accessible to bind the oligosaccharides of the recombinant glycoproteins.

The highest affinity in all cases was observed between MBL and PSGL-1/mIgG_{2b}. The three CRDs of MBL are arranged in a triangular fashion separated by approximately 54 Å suggesting that MBL is particularly suited to bind surfaces with repeating sugar groups of microbial origin [17]. The PSGL-1 parts of the dimeric PSGL-1/mIgG_{2b} are at least 200 Å and should with high substitution of oligosaccharides be able to present suitable ligands at appropriate distances to bind all three CRDs of MBL. Without intending to be bound by any theory, the fact that DC-SIGN binds AGP-1/mIgG_{2b} stronger than MBL in spite of having only a single CRD suggests a particularly strong interaction between DC-SIGN and its ligand. This also suggests that MBL may have a higher affinity for the terminal mannoses of the O-glycans of PSGL-1/mIgG_{2b} in contrast to the terminal mannoses of the N-glycans of AGP-1/mIgG_{2b}. Without intending to be bound by any theory, this could possibly also be related to the arrangement of the N-glycans of the two recombinant glycoproteins.

Carbohydrate Substitution of PSGL-1/mIgG_{2b} and AGP-1/mIgG_{2b} in Relation to Receptor Binding

AGP-1/mIgG_{2b} has a globular structure and has the potential to carry twelve N-linked glycans which upon expression in P. pastoris should be of the high mannose type (Man₈₋₁₄GlcNAc₂-Asn). It has been suggested that DC-SIGN has specificity towards the branch point trisaccharide Manα1-3[Manα1-6]Man in a configuration found only in N-linked high mannose oligosaccharides [13]. The results from the Biacore analyses supports this theory as DC-SIGN was shown to bind to AGP-1/mIgG_{2b} with the highest affinity of the different receptors. N-glycans derived from P. pastoris also provide terminal mannoses which have been shown to be ligands of MMR [14]. MBL, by virtue of its name, also selectively binds mannose but also other sugars like GlcNAc which is a part of N-glycans [19]. Thus, AGP-1/mIgG_{2b} should with its twelve N-glycans provide suitable ligands in multiple copies for all receptors facilitating multivalent binding. Specific geometrical and spatial orientations of the N-glycans could also provide certain molecular patterns with increased affinity to the receptors. This would, in part, explain the general high affinity binding between AGP-1/mIgG_{2b} and the receptors.

The carbohydrate analyses of PSGL-1/mIgG_{2b} described in Example 1 indicates that it carries both O- and N-linked oligosaccharides. Tandem mass spectrometry demonstrated that the O-glycans are linear structures with 2-9 hexoses. These results were surprising as O-glycans derived from P. pastoris which are composed of nine residues have never been characterized prior to the instant invention. Without intending to be bound by theory, based on previous studies of P. pastoris derived O-glycans and the fact that PSGL-1/mIgG_{2b} binds to Con A, the O-glycans should consist of mannose units exclusively [23]. Assuming that some or all six potential sites for N-glycosylation are also occupied, PSGL-1/mIgG_{2b} can provide multiple copies of ligands suitable for all receptors as well. The high affinity binding between DC-SIGN and PSGL-1/mIgG_{2b} supports the assumption that PSGL-1/mIgG_{2b} also carries N-glycans as the O-glycans do not carry the trisaccharide Manα1-3[Manα1-6]Man found in high mannose N-glycans which DC-SIGN is supposed to be specific for. Multiple N-glycans and the potential high number of O-glycans of variable length should promote the formation of clustered patches with enhanced receptor binding properties which may contribute to the high affinity binding. The role of the N-glycans of PSGL-1/mIgG_{2b} for binding DC-SIGN or the other receptors could be further investigated by specifically removing the N-glycans by PNGase F treatment before Biacore analysis.

As shown herein, PSGL-1/mIgG_{2b} generally binds with higher affinity to all receptors compared to AGP-1/mIgG_{2b}. A distinguishing feature of PSGL-1/mIgG_{2b} is its potential to present a large number of O-glycans on an extended polypeptide core. Without intending to be bound by any theory, the high numbers of oligosaccharides of various lengths probably have a better chance of mediating multivalent binding to several CRDs of the receptors where possible, but also to engage a larger number of non-specific binding outside the basic sugar binding site compared to AGP-1/mIgG_{2b}. This is under the presumption that PSGL-1/mIgG_{2b} expressed in P. pastoris has high site occupancy.

The molecular weight of PSGL-1/mIgG_{2b} without sugar substitution is approximately 117 kDa as a dimer. The typical molecular weight of PSGL-1/mIgG_{2b} expressed by P. pastoris was approximately 250-300 kDa for both bioreactor and shake flask cultivations (see Example 1). Without intending to be bound by theory, considering the sizes of the O- and N-glycans a reasonable explanation may be that a large fraction of the potential glycosylation sites are occupied. Without intending to be bound by any theory, the elongated structure of PSGL-1/mIgG_{2b} may also be an advantage for presenting its presumably densely packed oligosaccharides to the receptors which are located, in the case of Biacore analyses, on a flat surface or, in vivo, in cell microdomains. The spatial arrangement of the N-glycans on the globular structure of AGP-1/mIgG_{2b} may prevent neighbouring oligosaccharides to bind to the receptor, especially when they are immobilised on a flat surface as is the case for the Biacore experiments.

### Example 3

### Humanize the Repertoire of O-Glycans Produced by the Yeast Pichia Pastoris

The next step will be to express PSGL-1/mIgG_{2b} with a humanized O-glycan repertoire. To this end, we will co-express one or several UDP-N-acetyl-D-galactosaminide:polypeptide N-acetylgalactosaminyltransferases (ppGalNAc-Ts), which are the enzymes that in a peptide sequence-specific manner adds N-acetylgalactosamine residues to the amino acids serine or threonine in the peptide chain. Initially we will express the native forms of the enzymes. If this results in incorrect ER/Golgi localization, we will express chimeric forms of the enzymes in which the catalytic domain of the ppGalNAc-T has been fused to the transmembrane domain of the yeast-specific mannosyltransferase that links the first mannose residue to the peptide chain. If this does not work, transmembrane signal sequences from other type II proteins in Pichia will be tried. In addition, we most likely need to silence the expression of various mannosyltransferases involved in the biosynthesis of Pichia O-glycans. If a complete silencing through homologous recombination is lethal, we will try to accomplish a partial gene silencing using the siRNA technology. A partial silencing of the endogenous mannosyltransferases may with preserved yeast viability shift the equilibrium enough to favour the transfer of GalNAc residues instead of mannose residues. Further, to obtain a human-like O-glycan repertoire in Pichia it may also be necessary to express the transporter that takes UDP-GalNAc across the Golgi membrane. Mutant yeast colonies carrying human glycosyltransferases will be identified by lectin blots. In brief, replicas of the growing yeast colonies will be made by overlaying them with nitrocellulose membranes in order to capture secreted PSGL-1/mIgG fusion proteins. Following washing, the membranes will be probed with lectins of known carbohydrate specificity. Yeast colonies with the desired glycans on the PSGL-1 Ig fusion will be further expanded, and the O-glycan repertoire carried by the fusion protein will be structurally characterized following its purification. The recombinant protein is purified and structurally characterized as described above. If the initiating glycosylation step is successful, the innermost sugar can be built upon by introducing additional glycosyltransferase genes such that epitopes of therapeutic potential can be made.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

[1] V. Schijns, Mechanisms of vaccine adjuvant activity: initiation and regulation of immune responses by vaccine adjuvants, Vaccine 21 (2003) 829-831.
[2] V. Apostolopoulos, N. Barnes, G. A. Pietersz, I. F. C. McKenzie, Ex vivo targeting of the macrophage mannose receptor generates anti-tumor CTL responses, Vaccine 18 (2000) 3174-3184.
[3] V. Apostolopoulos, G. A. Pietersz, I. F. C. McKenzie, Cell-mediated immune responses to MUC1 fusion protein coupled to mannan, Vaccine 14 (1996) 930-938.
[4] A. J. Engering, M. Cella, D. Fluitsma, M. Brockhaus, E. C. M. Hoefsmit, A. Lanzavecchia, J. Pieters, The mannose receptor functions as a high capacity and broad specificity antigen receptor in human dendritic cells, European Journal of Immunology 27 (1997) 2417-2425.
[5] T. R. Gemmill, R. B. Trimble, Overview of N- and O-linked oligosaccharide structures found in various yeast species, Biochimica Et Biophysica Acta-General Subjects 1426 (1999) 227-237.
[6] J. S. Lam, M. K. Mansour, C. A. Specht, S. M. Levitz, A model vaccine exploiting fungal mannosylation to increase antigen immunogenicity, Journal of Immunology 175 (2005) 7496-7503.
[7] M. Luong, J. S. Lam, J. M. Chen, S. M. Levitz, Effects of fungal N- and O-linked mannosylation on the immunogenicity of model vaccines, Vaccine 25 (2007) 4340-4344.
[8] A. Engering, T. B. H. Geijtenbeek, S. J. van Vliet, M. Wijers, E. van Liempt, N. Demaurex, A. Lanzavecchia, J. Fransen, C. G. Figdor, V. Piguet, Y. van Kooyk, The dendritic cell-specific adhesion receptor DC-SIGN internalizes antigen for presentation to T cells, Journal of Immunology 168 (2002) 2118-2126.
[9] T. Keler, V. Ramakrishna, M. W. Fanger, Mannose receptor-targeted vaccines, Expert Opinion on Biological Therapy 4 (2004) 1953-1962.
[10] L. East, C. M. Isacke, The mannose receptor family, Biochimica Et Biophysica Acta-General Subjects 1572 (2002) 364-386.
[11] M. E. Taylor, K. Bezouska, K. Drickamer, Contribution to Ligand-Binding by Multiple Carbohydrate-Recognition Domains in the Macrophage Mannose Receptor, Journal of Biological Chemistry 267 (1992) 1719-1726.
[12] C. Y. Zhou T, Hao L, Zhang Y, DC-SIGN and Immunoregulation, Cellular and Molelcular Immunology 3 (2006) 279-283.
[13] H. Feinberg, D. A. Mitchell, K. Drickamer, W. I. Weis, Structural basis for selective recognition of oligosaccharides by DC-SIGN and DC-SIGNR, Science 294 (2001) 2163-2166.
[14] P. D. Stahl, The Macrophage Mannose Receptor—Current Status, American Journal of Respiratory Cell and Molecular Biology 2 (1990) 317-318.
[15] M. Chieppa, G. Bianchi, A. Doni, A. Del Prete, M. Sironi, G. Laskarin, P. Monti, L. Piemonti, A. Biondi, A. Mantovani, M. Introna, P. Allavena, Cross-linking of the mannose receptor on monocyte-derived dendritic cells activates an anti-inflammatory immunosuppressive program, Journal of Immunology 171 (2003) 4552-4560.
[16] S. Gordon, S. Clarke, D. Greaves, A. Doyle, Molecular Immunobiology of Macrophages—Recent Progress, Current Opinion in Immunology 7 (1995) 24-33.
[17] W. I. Weis, K. Drickamer, Trimeric Structure of a C-Type Mannose-Binding Protein, Structure 2 (1994) 1227-1240.
[18] M. Kuhlman, K. Joiner, R. A. B. Ezekowitz, The Human Mannose-Binding Protein Functions as an Opsonin, Journal of Experimental Medicine 169 (1989) 1733-1745.
[19] M. W. Turner, Mannose-binding lectin: The pluripotent molecule of the innate immune system, Immunology Today 17 (1996) 532-540.
[20] D. A. Fraser, A. J. Tenner, Directing an appropriate immune response: The role of defense collagens and other soluble pattern recognition molecules, Current Drug Targets 9 (2008) 113-122.
[21] R. K. Bretthauer, F. J. Castellino, Glycosylation of Pichia pastoris-derived proteins, Biotechnology and Applied Biochemistry 30 (1999) 193-200.
[22] J. G. Duman, R. G. Miele, H. Liang, D. K. Grella, K. L. Sim, F. J. Castellino, R. K. Bretthauer, O-Mannosylation of Pichia pastoris cellular and recombinant proteins, Biotechnology and Applied Biochemistry 28 (1998) 39-45.
[23] R. B. Trimble, C. Lubowski, C. R. Hauer, R. Stack, L. McNaughton, T. R.
Gemmill, S. A. Kumar, Characterization of N- and O-linked glycosylation of recombinant human bile salt-stimulated lipase secreted by Pichia pastoris, Glycobiology 14 (2004) 265-274.
[24] R. B. Trimble, P. H. Atkinson, J. F. Tschopp, R. R. Townsend, F. Maley, Structure of Oligosaccharides on Saccharomyces Suc2 Invertase Secreted by the Methylotrophic Yeast Pichia-Pastoris, Journal of Biological Chemistry 266 (1991) 22807-22817.
[25] A. Gustafsson, Holgersson J, A new generation of carbohydrate based therapeutics: recombinant mucin-type fusion proteins as versatile inhibitors of protein-carbohydrate interactions, Expert opinion on Drug Discovery 1 (2006) 161-178.
[26] M. J. Treuheit, C. E. Costello, H. B. Halsall, Analysis of the 5 Glycosylation Sites of Human Alpha-1-Acid Glycoprotein, Biochemical Journal 283 (1992) 105-112.
[27] J. N. Liu, Y. J. Qian, J. Holgersson, Removal of xenoreactive human anti-pig antibodies by absorption on recombinant mucin-containing glycoproteins carrying the Gal alpha 1,3Gal epitope, Transplantation 63 (1997) 1673-1682.
[28] J. Holgersson, J. Lofling, Glycosyltransferases involved in type 1 chain and Lewis antigen biosynthesis exhibit glycan and core chain specificity, Glycobiology 16 (2006) 584-593.
[29] J. N. Liu, A. Gustafsson, M. E. Breimer, A. Kussak, J. Holgersson, Anti-pig antibody adsorption efficacy of alpha-Gal carrying recombinant P-selectin glycoprotein ligand-1/immunoglobulin chimeras increases with core 2 beta 1,6-N-acetylglucosaminyltransferase expression, Glycobiology 15 (2005) 571-583.
[30] I. Carlstedt, A. Herrmann, H. Karlsson, J. Sheehan, L. A. Fransson, G. C. Hansson, Characterization of 2 Different Glycosylated Domains from the Insoluble Mucin Complex of Rat Small-Intestine, Journal of Biological Chemistry 268 (1993) 18771-18781.
[31] I. Ciucanu, F. Kerek, A simple and rapid method for the permethylation of carbohydrates, Carbohydrate research 131 (1984) 209-217.
[32] G. C. Hansson, H. Karlsson, Gas chromatography and gas chromatography-mass spectrometry of glycoprotein oligosaccharides, Methods in Molecular Biology 14 (1993) 47-54.
[33] J. N. Liu, A. Weintraub, J. Holgersson, Multivalent Gal alpha 1,3Gal-substitution makes recombinant mucin-immunoglobulins efficient absorbers of anti-pig antibodies, Xenotransplantation 10 (2003) 149-163.
[34] A. B. Rawitch, H. G. Pollock, S. X. Yang, Thyroglobulin Glycosylation—Location and Nature of the N-Linked Oligosaccharide Units in Bovine Thyroglobulin, Arch. Biochem. Biophys. 300 (1993) 271-279.
[35] D. A. Mitchell, A. J. Fadden, K. Drickamer, A novel mechanism of carbohydrate recognition by the C-type lectins DC-SIGN and DC-SIGNR—Subunit organization and binding to multivalent ligands, Journal of Biological Chemistry 276 (2001) 28939-28945.
[36] K. E. Norgard, K. L. Moore, S. Diaz, N. L. Stults, S. Ushiyama, R. P. McEver, R. D. Cummings, A. Varki, Characterization of a Specific Ligand for P-Selectin on Myeloid Cells—a Minor Glycoprotein with Sialylated O-Linked Oligosaccharides, Journal of Biological Chemistry 268 (1993) 12764-12774.
[37] M. Jahic, A. Veide, T. Charoenrat, T. Teeri, S. O. Enfors, Process technology for production and recovery of heterologous proteins with Pichia pastoris (vol 22, pg 1472, 2006), Biotechnology Progress 23 (2007) 516-516.
[38] T. Charoenrat, M. Ketudat-Caims, H. Stendahl-Andersen, M. Jahic, S. O. Enfors, Oxygen-limited fed-batch process: an alternative control for Pichia pastoris recombinant protein processes, Bioprocess and Biosystems Engineering 27 (2005) 399-406.
[39] S. O. Enfors, M. Jahic, A. Rozkov, B. Xu, M. Hecker, B. Jurgen, E. Kruger, T. Schweder, G. Hamer, D. O'Beirne, N. Noisommit-Rizzi, M. Reuss, L. Boone, C. Hewitt, C. McFarlane, A. Nienow, T. Kovacs, C. Tragardh, L. Fuchs, J. Revstedt, P. C. Friberg, B. Hjertager, G. Blomsten, H. Skogman, S. Hjort, F. Hoeks, H. Y. Lin, P. Neubauer, R. van der Lans, K. Luyben, P. Vrabel, A. Manelius, Physiological responses to mixing in large scale bioreactors, Journal of Biotechnology 85 (2001) 175-185.
[40] H. J. Li, N. Sethuraman, T. A. Stadheim, D. X. Zha, B. Prinz, N. Ballew, P. Bobrowicz, B. K. Choi, W. J. Cook, M. Cukan, N. R. Houston-Cummings, R. Davidson, B. Gong, S. R. Hamilton, J. P. Hoopes, Y. W. Jiang, N. Kim, R. Mansfield, J. H. Nett, S. Rios, R. Strawbridge, S. Wildt, T. U. Gemgross, Optimization of humanized IgGs in glycoengineered Pichia pastoris, Nature Biotechnology 24 (2006) 210-215.
[41] D. T. Y. Liu, Glycoprotein Pharmaceuticals—Scientific and Regulatory Considerations, and the United-States Orphan Drug-Act, Trends in Biotechnology 10 (1992) 114-120.
[42] J. L. Cereghino, J. M. Cregg, Heterologous protein expression in the methylotrophic yeast Pichia pastoris, Fems Microbiology Reviews 24 (2000) 45-66.
[43] M. Jahic, F. Wallberg, M. Bollok, P. Garcia, S.-O. Enfors, Temperature limited fed-batch technique for control of proteolysis in Pichia pastoris bioreactor cultures, Microbial Cell Factories 2 (2003) 6.
[44] Y. W. Zhang, R. J. Liu, X. Y. Wu, The proteolytic systems and heterologous proteins degradation in the methylotrophic yeast Pichia pastoris, Annals of Microbiology 57 (2007) 553-560.
[45] C. Goochee, ENVIRONMENTAL-EFFECTS ON PROTEIN GLYCOSYLATION, Bio/technology 8 (1990) 421-427.

What is claimed is:

1. A fusion polypeptide comprising a first polypeptide operably linked to a second polypeptide wherein the first polypeptide is mannosylated and the second polypeptide comprises at least a region of an immunoglobulin polypeptide.

2. The fusion polypeptide of claim 1, wherein the first polypeptide is a mucin polypeptide.

3. The fusion polypeptide of claim 2, wherein the mucin is selected from the group consisting of PSGL-1, MUC1, MUC2, MUC3a, MUC3b, MUC4, MUC5a, MUC5b, MUC5c, MUC6, MUC10, MUC11, MUC12, MUC13, MUC15, MUC16, MUC17, CD34, CD43, CD45, CD96, GlyCAM-1, MAdCAM, or a fragment thereof

4. The fusion polypeptide of claim 2, wherein said mucin polypeptide comprises at least a region of a P-selectin glycoprotein ligand-1.

5. The fusion polypeptide of claim 2, wherein said mucin polypeptide includes an extracellular region of a P-selectin glycoprotein ligand-1.

6. The fusion polypeptide of claim 1, wherein the first polypeptide is an alpha glycoprotein polypeptide.

7. The fusion polypeptide of claim 1, wherein the first polypeptide comprises at least a region of an alpha-1-acid glycoprotein.

8. The fusion polypeptide of any of claims 2-7, wherein the fusion polypeptide binds to a mannose-binding receptor with higher affinity than a wild type mucin or alpha glycoprotein polypeptide.

9. The fusion polypeptide of claim 8, wherein the fusion polypeptide binds to a mannose-binding receptor with an affinity ranging from 1 nM to 100 nM.

10. The fusion polypeptide of claim 8, wherein said mannose-binding receptor is selected from the group consisting of macrophage mannose receptor (MMR), dendritic cell-specific ICAM-3-grabbing non-integrin (DC-SIGN) and mannose binding lectin (MBL).

11. The fusion polypeptide of claim 10, wherein the mannose-binding receptor is MMR and the fusion polypeptide binds to MMR with an affinity of 70-90 nM.

12. The fusion polypeptide of claim 10, wherein the mannose-binding receptor is DC-SIGN and the fusion polypeptide binds to DC-SIGN with an affinity of 5 nM to 25 nM.

13. The fusion polypeptide of claim 10, wherein the mannose-binding receptor is MBL, and the fusion polypeptide binds to MBL with an affinity of 1 nM to 50 nM.

14. The fusion polypeptide of claim 1, wherein the second polypeptide comprises a region of a heavy chain immunoglobulin polypeptide.

15. The fusion polypeptide of claim 1, wherein said second polypeptide comprises an F_{c} region of an immunoglobulin heavy chain.

16. An adjuvant composition comprising the fusion polypeptide of claim 1.

17. A method of vaccinating a subject in need thereof comprising administering the subject a composition comprising the adjuvant of claim 16 and an antigen.

18. A yeast cell genetically engineered to produce the fusion polypeptide of claim 1.

19. The yeast cell of claim 18, wherein said cell is Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pyperi, Pichia stiptis, Pichia methanolica, Pichia sp., Saccharomyces cerevisiae, Saccharomyces sp., Hansenulapolymorpha, Kluyveromyces sp., Candida albicans, Aspergillus nidulans, or Trichoderma reesei.

20. A genetically engineered lower eukaryotic cell producing human-like glycoproteins characterized as having O-linked glycans.

21. The cell of claim 20, where the cell expresses N-acetylgalactosaminyltransferase(s).

22. A recombinant lower eukaryotic cell producing human-like glycoproteins wherein said cell comprises a nucleic acid molecule encoding N-acetylgalactosaminyltransferase(s).

23. The cell of claim 20 or 22, wherein said cell is Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pyperi, Pichia stiptis, Pichia methanolica, Pichia sp., Saccharomyces cerevisiae, Saccharomyces sp., Hansenulapolymorpha, Kluyveromyces sp., Candida albicans, Aspergillus nidulans, or Trichoderma reesei.

24. The cell of claim 20 or 22, wherein said cell expresses an eliminated or reduced level of one or more enzymes involved in production of N-glycans or O-glycans.

25. A method of producing a mannosylated polypeptide having increased glycosylation as compared to a wild type polypeptide comprising:
a) introducing into a lower eukaryotic cell
i) a nucleic acid encoding said polypeptide; and
b) culturing the cell under conditions that permit cell growth;
c) inducing production of said polypeptide by said cells and culturing said cells under conditions which permit increased glycosylation and decreased fragmentation of said polypeptide; and
d) isolating said polypeptide.

26. The method of claim 25, wherein the cell is cultured in a fermentor (bioreactor).

27. The method of claim 25, wherein the cell culture conditions which permit cell growth in step b) comprise 29° C. and pH 6.0.

28. The method of claim 25, wherein the cell culture conditions which permit increased glycosylation and decreased fragmentation of said polypeptide in step c) comprise a lower pH than the culture conditions which permit cell growth and 29° C.

29. The method of claim 25, wherein said polypeptide production is induced in step c) using methanol.

30. A method of producing a mannosylated polypeptide having increased glycosylation and decreased fragmentation as compared to a wild type polypeptide comprising:
a) providing a cell culture having a neutral or near neutral pH of a lower eukaryotic cell comprising a nucleic acid enconding said polypeptide;
b) inducing production of said polypeptide by said cells under conditions which permit increased glycosylation and decreased fragmentation of said polypeptide, wherein said conditions comprise a lower pH than the cell culture of step a); and
c) isolating said polypeptide.

31. A polypeptide produced according to the method of claim 25 or 30.

32. The method of claim 31, wherein said polypeptide is a mucin polypeptide or an alpha glycoprotein polypeptide.


SEQUENCE LISTING (4 sequences)

SEQ ID NO: 1 - DNA, 30 bp, Artificial Sequence
  Synthetic oligonucleotide
cgcgggaattccagctgtgggacacctggg

SEQ ID NO: 2 - DNA, 35 bp, Artificial Sequence
  Synthetic oligonucleotide
gcgggaattctcatttacccggagaccgggagatg

SEQ ID NO: 3 - DNA, 23 bp, Artificial Sequence
  Synthetic oligonucleotide
cgcgggaattccagatcccattg

SEQ ID NO: 4 - DNA, 35 bp, Artificial Sequence
  Synthetic oligonucleotide
gcggggtacctcatttacccggagaccgggagatg